SEQUENCE LISTING (14 sequences)

SEQ ID NO: 1 - DNA, 20 bp, Artificial Sequence
  synthetic primer
ggttgctacgtccttctgga

SEQ ID NO: 2 - DNA, 20 bp, Artificial Sequence
  synthetic primer
tgtgcctcctccgatatctt

SEQ ID NO: 3 - DNA, 22 bp, Artificial Sequence
  synthetic primer
ccgtggacgtgatgtaccgcac

SEQ ID NO: 4 - DNA, 21 bp, Artificial Sequence
  synthetic primer
cgcaatgggcatgcgtctctg

SEQ ID NO: 5 - DNA, 21 bp, Artificial Sequence
  synthetic primer
cgcaatgggcatgcgtctctg

SEQ ID NO: 6 - DNA, 20 bp, Artificial Sequence
  synthetic primer
ctccagcatgaggtgagtgt

SEQ ID NO: 7 - DNA, 20 bp, Artificial Sequence
  synthetic primer
gcttctttgttcaccgcttc

SEQ ID NO: 8 - DNA, 20 bp, Artificial Sequence
  synthetic primer
gcttgaccagcttcagttcc

SEQ ID NO: 9 - DNA, 20 bp, Artificial Sequence
  synthetic primer
agtcccaaaagcaaacgaag

SEQ ID NO: 10 - DNA, 19 bp, Artificial Sequence
  synthetic primer
ttgtccaaatcaccgatgc

SEQ ID NO: 11 - DNA, 21 bp, Artificial Sequence
  synthetic primer
ttgtctgggcaagaggatcag

SEQ ID NO: 12 - DNA, 21 bp, Artificial Sequence
  synthetic primer
accactcgcacttgcactttc

SEQ ID NO: 13 - DNA, 20 bp, Artificial Sequence
  synthetic primer
agagtttgatcctggctcag

SEQ ID NO: 14 - DNA, 15 bp, Artificial Sequence
  synthetic primer
ctgctgcctyccgta

(19) United States
(12) Patent Application Publication
(10) Pub. No.: US 2024/0000753 A1
(43) Pub. Date: Jan. 4, 2024

(54) METHODS AND COMPOSITIONS TO INCREASE LIFESPAN AND HEALTHSPAN BY MIMICKING THE EFFECTS OF TIME-RESTRICTED FEEDING

(71) Applicant: The Trustees of Columbia University in the City of New York, New York, NY (US)

(72) Inventors: Mimi Shirasu-Hiza, New York, NY (US); Julie Canman, New York, NY (US); Matt Ulgherait, New York, NY (US)

(21) Appl. No.: 18/016,516

(22) PCT Filed: Jul. 19, 2021
(86) PCT No.: PCT/US2021/042146

§ 371 Date: Jan. 17, 2023

Related U.S. Application Data
(60) Provisional application No. 63/053,037, filed on Jul. 17, 2020.

(51) Int. Cl. A61K 31/4188; C12N 15/63; A61P 9/00; A61K 31/155; A61K 31/496; A61K 31/18; A61K 31/436

(52) U.S. Cl. CPC A61K 31/4188; C12N 15/63; A61P 9/00; A61K 31/155; A61K 31/496; A61K 31/18; A61K 31/436

(57) ABSTRACT

The present disclosure relates to methods and compositions for increasing or extending lifespan and/or healthspan and/or delaying aging using agents which mimic the effects of time-restricted feeding and/or activate or enhance circadian-regulated autophagy. In particular, the present disclosure relates to increasing certain proteins including UNC-51-like kinase (ULK1), adenosine monophosphate protein kinase (AMPK) and microtubule-associated protein, light chain 3 (LC3), and decreasing other certain proteins including ribosomal protein S6 kinase beta-1 (S6K) protein in order to increase lifespan and/or healthspan and delay aging.

### CROSS REFERENCE TO RELATED APPLICATION

The present application claims priority to U.S. Patent Application Ser. No. 63/053,037 filed Jul. 17, 2020, which is hereby incorporated by reference in its entirety.

### STATEMENT OF GOVERNMENT INTEREST

This invention was made with government support under through grants R01GM117407, R35GM127049, R56AG065986, R01GM130764 and R01AG045842, all awarded by the National Institutes of Health. The government has certain rights in the invention.

### FIELD

The present disclosure relates to methods and compositions for increasing or extending lifespan and/or healthspan and/or delaying aging by using agents which mimic the effects of time-restricted feeding and/or activate or enhance circadian-regulated autophagy. In particular, the present disclosure relates to increasing certain proteins including UNC-51-like kinase (ULK1), adenosine monophosphate protein kinase (AMPK) and microtubule-associated protein, light chain 3 (LC3), and decreasing other certain proteins including ribosomal protein S6 kinase beta-1 (S6K) protein in order to increase lifespan and/or healthspan and/or delay aging.

### BACKGROUND

Time-restricted feeding (TRF) has become a potential anti-aging treatment of high interest in recent years, with the ability to delay aging and improve health in diverse organisms from Drosophila to humans (Longo and Panda 2016; Villanueva et al. 2019; Chaix et al. 2014; Gill et al. 2015). TRF consists of restricting food intake to specific hours of the day. Because TRF controls the timing of feeding rather than nutrient or caloric content, TRF has been hypothesized to depend on circadian-regulated functions. TRF has been mainly studied in mammals, however, the underlying molecular mechanisms remain unclear.

TRF in humans otherwise known as intermittent fasting, while popular, is difficult for many to follow. If the underlying molecular mechanisms, including the pathways and proteins which are targeted by time-restricted feeding, can be elucidated, the benefits of time-restricted feeding can be enjoyed by people without the burden of following a time-restricted feeding schedule.

### SUMMARY

As shown herein, time-restricted feeding or fasting increased lifespan as well as healthspan and delayed aging. Moreover, the increase in lifespan and healthspan was due in part to the activation or increase in circadian-regulated autophagy. The proteins which were involved in this process included Drosophila Atg1 and Atg8, which correspond to human UNC-51-like kinase (ULK1) and microtubule-associated protein, light chain 3 (LC3). Additionally, proteins adenosine monophosphate protein kinase (AMPK) and ribosomal protein S6 kinase beta-1 (S6K) were targeted by the time-restricted feeding.

Targeting these proteins and the genes which encode them, can allow the benefits of time-restricted feeding to be gained in a subject without the burden of following a time-restricted feeding schedule.

Thus, one embodiment of the present disclosure is a method of increasing and/or extending lifespan and/or healthspan and/or delaying aging in a subject by administering a therapeutically effective amount of an agent which mimics the effects of time-restricted feeding.

A further embodiment of the present disclosure is a method of increasing and/or extending lifespan and/or healthspan and/or delaying aging in a subject by administering a therapeutically effective amount of an agent which activates circadian-regulated autophagy.

Agents which can be use in the methods provided herein include but are not limited to chemicals, pharmaceuticals, biologics, small organic molecules, antibodies, nucleic acids, peptides, and proteins.

A further embodiment of the present disclosure is a method of increasing and/or extending lifespan and/or healthspan and/or delaying aging in a subject by administering a therapeutically effective amount of an agent which overexpresses, increases, activates or enhances a protein, wherein the overexpression, increase, activation or enhancement of the protein mimics the effects of time-restricted feeding.

A further embodiment of the present disclosure is a method of increasing and/or extending lifespan and/or healthspan and/or delaying aging in a subject by administering a therapeutically effective amount of an agent which overexpresses, increases, activates or enhances a protein, wherein the overexpression, increase, activation or enhancement of the protein activates circadian-regulated autophagy

Proteins which can be targeted by these methods include but are not limited to ULK1, AMPK, and LC3.

In some embodiments of the foregoing methods, the agent is the protein itself or a nucleic acid encoding a protein or a variant, mutant, fragment, homologue, or derivative thereof.

In some embodiments of the foregoing methods, the agent is a pharmaceutical or small molecule, including but not limited to AICAR, Metformin HC1, LYN-1604, BL-918 and (rac)-BL-919.

Yet a further embodiment of the present disclosure is a method of increasing and/or extending lifespan and/or healthspan and/or delaying aging in a subject by administering a therapeutically effective amount of an agent which inhibits a protein, wherein the inhibition of the protein mimics the effects of time-restricted feeding.

Yet a further embodiment of the present disclosure is a method of increasing and/or extending lifespan and/or healthspan and/or delaying aging in a subject by administering a therapeutically effective amount of an agent which inhibits a protein, wherein the inhibition of the protein activates circadian-regulated autophagy.

Proteins which can be targeted by these methods include but are not limited to S6K protein.

In one embodiment of the foregoing method, agents that can be used to inhibit S6K protein include but are not limited to pharmaceuticals, small molecules, nucleic acids, proteins, and antibodies. In some embodiments, the nucleic acid includes but is not limited to antisense oligonucleotide, a small interfering RNA (siRNA), a short hairpin RNA (shRNA), a guide RNA (gRNA), aptamer, and combinations thereof. In some embodiments of the foregoing methods, the agent is a pharmaceutical or small molecule, including but not limited rapamycin.

In some embodiments, the subject is healthy. In some embodiments, the subject is elderly. In some embodiments, the subject is suffering from age-related illness. In some embodiments, the subject is having symptoms of decreased healthspan or aging including but not limited to protein aggregation in muscles and intestinal leakage in the gut

In some embodiments, the subject is under 50 years of age. In some embodiments, the subject is over 50 years of age. In some embodiments, the subject is under 65 years of age. In some embodiments, the subject is over 65 years of age. In some embodiments, the subject is under 70 years of age. In some embodiments, the subject is over 70 years of age. In some embodiments, the subject is under 75 years of age. In some embodiments, the subject is over 75 years of age. In some embodiments, the subject is under 80 years of age. In some embodiments, the subject is over 80 years of age. In some embodiments, the subject is under 85 years of age. In some embodiments, the subject is over 85 years of age. In some embodiments, the subject is under 90 years of age. In some embodiments, the subject is over 90 years of age.

In some embodiments, the agent is administered at night.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1. Lifespan changes in response to different feeding and fasting regimens. FIG. 1A is the schematic of different feeding regimens utilized in lifespan screen. FIG. 1B shows the results of 12-hour time-restricted feeding (TRF) from day 10 until death shortens female lifespan (top) and minimally extends male lifespan (bottom). FIG. 1C shows results of the TRF diet regimen executed between days 10-40 extends female (top) and male (bottom) lifespan. FIG. 1D shows the results of the 24-hour intermittent fasting shortens both female (top) and male (bottom) lifespan when executed day 10 until death. FIG. 1E shows the percent survival of flies on the iTRF diet relative to ad lib diet. FIG. 1F shows the 10-day windows of iTRF in early to mid-life can extend male and female mean lifespan by approximately 10% compared to ad lib, while longer administration of iTRF 10-40 extends mean lifespan of flies by 15-18%. n.s.=p>0.05, *=p<0.05, **=p<0.01, ***=p<0.001; p-values were obtained by unpaired two-tailed t-test (D, G), ANOVA followed by Tukey's post-hoc test (C, F), and log-rank analysis (B, E). Error bars represent SEM.

FIG. 2. Intermittent Time-Restricted Feeding (iTRF) extends lifespan and healthspan and is not due to dietary restriction. FIG. 2A shows food intake of each group of flies the showing that the iTRF flies starve during the fast phase (blue dots), eat more during the feeding phase (green dots). FIG. 2B shows the total food intake for each group of flies. FIG. 2C shows the lifespan of the iTRF and dietary protein restriction (DR) flies and that iTRF and DR extended lifespan additively. FIG. 2D is a graph of lifespan of iTRF and ad lib flies with and without partial genetic ablation of insulin producing cells. n.s.=p>0.05, *=p<0.05, **=p<0.01, ***=p<0.001; p-values were obtained by unpaired two-tailed t-test (D, G), ANOVA followed by Tukey's post-hoc test (C, F), and log-rank analysis (B, E). Error bars represent SEM.

FIG. 3. Intermittent Time-Restricted Feeding (iTRF) extends lifespan, reduces accumulation of protein aggregates with age, and delays intestinal aging. FIG. 3A shows climbing activity in iTRF and ad lib flies and that iTRF decreased age-related declines in climbing activity. FIG. 3B is a graph of ubiquitin levels in iTRF and ad lib flies. FIG. 3C is a graph of the levels of p62 in iTRF and ad lib flies. FIG. 3D is a graph of accumulated polyubiquitin aggregate levels in iTRF and ad lib flies. FIG. 3E is a graph of the accumulated levels of p62 muscle aggregates in iTRF and ad lib flies. FIG. 3F shows the amounts of mitotic cells in the posterior gut of iTRF and ad lib flies. FIG. 3G is a graph of intestinal barrier function (day 10-40) in iTRF and ad lib flies. FIG. 3H is the bacterial load of iTRF and ad lib flies treated and not treated with antibiotics. iTRF flies showed delayed age-related microbiome load with age. FIG. 3I shows life span of iTRF and ad lib flies given antibiotics throughout life. FIG. 3J shows life spans of iTRF and ad lib flies given antibiotics day 10-40. n.s.=p>0.05, *=p<0.05, **=p<0.01, ***=p<0.001; p-values were obtained by unpaired two-tailed t-test (D, G), ANOVA followed by Tukey's post-hoc test (C, F), and log-rank analysis (B, E). Error bars represent SEM.

FIG. 4. Core circadian clock components are required for iTRF-mediated lifespan and healthspan extension. FIG. 4A is a schematic of the core transcriptional circadian clock. FIG. 4B is relative clock expression in flies on ad lib diet (solid gray lines) and iTRF (dashed gray lines). FIG. 4C is relative period expression in flies on ad lib diet (solid gray lines) and iTRF (dashed gray lines). FIG. 4D is relative timeless expression in flies on ad lib diet (solid gray lines) and iTRF (dashed gray lines). FIGS. 4B-4D shows that iTRF broadened the peak of clock expression and increased the amplitude of period and timeless expression during fasting. FIG. 4E shows the life span of iTRF and ad lib flies controls and cyc⁰¹ mutants (red orange). FIG. 4F shows the life span of iTRF and ad lib flies controls and per⁰¹ (orange). FIG. 4G shows the life span of iTRF and ad lib flies controls and tim^{CRISPR} (gold) and per^{CRISPR} (orange) mutants. FIG. 4H is a graph of life span of male iTRF and ad lib flies controls and per⁰¹. FIG. 4I is a graph of climbing activity in iTRF and ad lib flies in controls and per⁰¹ mutants (orange). FIG. 4J is a graph of the level of ubiquitin in iTRF and ad lib flies in controls and per⁰¹ mutants. FIG. 4K is a graph of the level of p62 in iTRF and ad lib flies in controls and per⁰¹ mutants. Relative to ad lib diet, iTRF inhibited two aging markers in controls but not perm . FIG. 4L is a schematic of a day biased/night biased feeding schedule. FIG. 4M are graphs showing the lifespan of night-biased iTRF (left, dashed gray, n=322), and day-biased iTRF (right, dashed gray, n=286) as compared to ad lib diet (solid gray, n=553); per01 mutants (orange) were not affected by night- or day-biased iTRF (n=209-218). n.s.=p>0.05, *=p<0.05, **=p<0.01, ***=p<0.001; ANOVA followed by Tukey's post-hoc test (B, C, D, I), and log-rank analysis (E-H, M). Error bars represent SEM.

FIG. 5. Key autophagy components are required for iTRF-mediated lifespan extension and healthspan extension. FIG. 5A is a schematic of core autophagy components. FIG. 5B is a graph of relative atg1 expression in iTRF and ad lib flies. FIG. 5C is a graph of relative atg8 expression in iTRF and ad lib flies. FIG. 5D is a graph of relative atg1 expression in iTRF and ad lib flies controls and per⁰¹. FIG. 5E is a graph of relative atg8 expression in iTRF and ad lib flies controls and per⁰¹. FIGS. 5B-5E show that both atg1 and atg8a expression were circadian-regulated; relative to ad lib diet (solid gray lines), iTRF (dashed gray lines) broadened the peak of atg1 expression and increased the amplitude of atg8a expression in wild-type flies and that iTRF (light shades) did not alter atg1 expression, atg8a expression, or lipidated Atg8 protein levels in per⁰¹ mutants (orange) relative to controls (gray) and ad lib diet (dark shades). FIG. 5F is a graph of p-AMPK levels in iTRF and ad lib flies controls and per⁰¹. FIG. 5G is a graph of p-56K levels in iTRF and ad lib flies controls and per⁰¹. FIG. 5H is a graph of number of autolysosomes in the intestines of controls (light gray) compared to per⁰¹ mutants (orange) as measured by staining. FIG. 5I is a graph of the life span of iTRF and ad lib flies controls and RNAi-mediated knockdown of atg1. FIG. 5J is a graph of the life span of iTRF and ad lib flies controls and RNAi-mediated knockdown of atg8. FIG. 5K is a graph of the life span of iTRF and ad lib flies treated with RU486 and controls. FIG. 5L is a graph of the life span of iTRF and ad lib flies in overexpressed atg1 in per⁰¹ mutants. n.s.=p>0.05, *=p<0.05, **=p<0.01, ***=p<0.001; p-values were obtained by unpaired two-tailed t-test (F), ANOVA followed by Tukey's post-hoc test (B-E), and log-rank analysis (G-I). Error bars represent SEM.

FIG. 6. Increasing circadian-regulated expression of autophagy-promoting genes is necessary and sufficient for the health benefits of iTRF. FIG. 6A is a schematic of genetic method to overexpress autophagy components with circadian rhythmicity. FIG. 6B is a graph of life span of ad lib flies with various circadian overexpression data. FIG. 6C is a graph of life span of iTRF flies with various circadian overexpression data. FIG. 6D is a graph of life span of control ad lib and iTRF flies. FIG. 6E is a graph of the life span of iTRF and ad lib flies with circadian knockdown of atg1. FIG. 6F is a graph of the life span of iTRF and ad lib flies with genetic circadian overexpression of atg1. FIG. 6G a graph of the life span of iTRF and ad lib flies with circadian knockdown of atg8 and shows that circadian knockdown of ATG8a is necessary for iTRF-mediated lifespan extension. FIG. 6H is a graph of the life span of iTRF and ad lib flies with genetic circadian overexpression of atg8 and shows that circadian overexpression of Atg8a is sufficient to extend lifespan on ad lib conditions and responds minimally to iTRF. FIG. 6I is a graph of the life span of iTRF and ad lib flies with pharmacologically induced circadian overexpression of atg1. FIG. 6J is a graph of climbing activity of iTRF and ad lib flies with circadian knockdown of atg1. FIG. 6K is a graph of ubiquitin levels of iTRF and ad lib flies with circadian knockdown of atg1. FIG. 6L is a graph of p62 levels of iTRF and ad lib flies with circadian knockdown of atg1. FIG. 6M is a graph of climbing activity of iTRF and ad lib flies with circadian overexpression of atg1. FIG. 6N is a graph of ubiquitin levels of iTRF and ad lib flies with circadian overexpression of atg1. FIG. 6O is a graph of p62 levels of iTRF and ad lib flies with circadian overexpression of atg1. FIG. 6P is a graph of the life span of iTRF and ad lib flies with pharmacologically induced circadian overexpression of atg1 during the night phase. FIG. 6Q is a graph of the life span of iTRF and ad lib flies with pharmacologically induced circadian overexpression of atg1 during the day phase. n.s.=p>0.05, *=p<0.05, **=p<0.01, ***=p<0.001; ANOVA followed by Tukey's post-hoc test (B-F), and log-rank analysis (H-J and P-Q). Error bars represent SEM.

### DETAILED DESCRIPTION

### Definitions

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The invention is particularly desirable for human medical applications.

A therapeutically effective amount, or an effective amount, of a drug or an agent is an amount effective to demonstrate a desired activity of the drug or the agent. A “therapeutically effective amount” will vary depending on the compound, the disorder and its severity and the age, weight, physical condition and responsiveness of the subject to be treated. In certain embodiments, a “therapeutically effective amount of an agent” is used herein to mean an amount sufficient to cause an improvement in a clinically significant condition or in the healthspan and/or lifespan in the subject, or delays or minimizes or mitigates one or more symptoms associated with the decreasing healthspan and/or lifespan, and/or delays aging and/or minimizes or mitigates one or more symptoms associated with aging or results in a desired beneficial change of physiology in the subject.

The term “agent” as used herein means a substance that produces or is capable of producing an effect and would include, but is not limited to, chemicals, pharmaceuticals, biologics, small organic molecules, antibodies, nucleic acids, peptides, and proteins.

The term “healthspan” is used to mean the part of a person's life during which they are generally in good health

The term “composition” as used herein means a product which results from the mixing or combining of more than one element or ingredient.

As used herein, the term “carrier” refers to a diluent, adjuvant, excipient, or vehicle with which the therapeutic is administered, and includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art.

The term “pharmaceutically-acceptable” refers to molecular entities and compositions that do not produce an allergic or similar untoward reaction when administered to a host, such as gastric upset, dizziness and the like, when administered to a human, and approved by a regulatory agency of the Federal or a state government or listed in the U.S. Pharmacopeia or other generally recognized pharmacopeia for use in animals, and more particularly in humans.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector” includes any genetic element, such as a plasmid, phage, transposon, cosmid, chromosome, artificial chromosome, virus, or virion, which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “operatively linked,” “under control,” or “under transcriptional control” means that the promoter is in the correct location and orientation in relation to the nucleic acid to control RNA polymerase initiation and expression of the gene.

The term “expression vector” or “expression construct” or “construct” means any type of genetic construct containing a nucleic acid in which part or all of the nucleic acid encoding sequence is capable of being transcribed. In some embodiments, expression includes transcription of the nucleic acid, for example, to generate a biologically active polypeptide product or inhibitory RNA from a transcribed gene.

With respect to transfected host cells, the term “transfection” is used to refer to the uptake of foreign DNA by a cell, and a cell has been “transfected” when exogenous DNA has been introduced inside the cell membrane. A number of transfection techniques are generally known in the art. See, e.g., Graham et al., Virology 52:456 (1973), Sambrook et al., Molecular Cloning, a Laboratory Manual, Cold Spring Harbor Laboratories, New York (1989), Davis et al., Basic Methods in Molecular Biology, Elsevier (1986), and Chu et al., Gene 13:197 (1981). Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically active polypeptide or production of a biologically active nucleic acid such as an RNA, has been introduced.

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Benefits and Targets of Intermittent Time-Restricted Feeding (iTRF)

To exploit its rapid genetic tools and well-characterized aging markers, an alternate-day, intermittent TRF (iTRF) dietary regimen was developed for Drosophila. As shown herein, iTRF robustly extended lifespan and delayed aging-dependent processes such as poly-ubiquitinated protein aggregation in muscles and intestinal leakage in the gut (examples of prolonged healthspan). Also shown herein is that the longevity effects of iTRF required molecular components of the circadian clock and, conversely, that iTRF treatment of animals enhanced circadian regulated transcription. Also shown herein is that iTRF depended on circadian regulated components of autophagy, a conserved longevity pathway. Furthermore, overexpressing autophagy components in a circadian manner was sufficient to extend lifespan, even with normal diet. Thus, these results identified an essential circadian regulated metabolic process that can be manipulated by a behavioral intervention to delay aging, improve healthspan and extend lifespan in Drosophila. Because both circadian regulation and autophagy are highly conserved processes that play roles in human aging, this work highlights the possibility that pharmaceutical interventions stimulating circadian-regulated autophagy may provide people with similar health benefits such as delayed aging and long life.

As shown herein the proteins human UNC-51-like kinase (ULK1), microtubule-associated protein, light chain 3A (LC3), as well as adenosine monophosphate protein kinase (AMPK) and ribosomal protein S6 kinase beta-1 (S6K) were targeted by the time-restricted feeding.

ULK1 is an enzyme that in humans is encoded by the ULK1 gene. It is specifically a kinase that is involved with autophagy, particularly in response to amino acid withdrawal.

LC3 is also an effector of autophagy.

AMPK is an enzyme that plays a role in cellular energy homeostasis, largely to activate glucose and fatty acid uptake and oxidation when cellular energy is low. It belongs to a highly conserved eukaryotic protein family and its orthologues are SNF1 in yeast, and SnRK1 in plants. It consists of three proteins (subunits) that together make a functional enzyme, conserved from yeast to humans. It is expressed in a number of tissues, including the liver, brain, and skeletal muscle. In response to binding AMP and ADP, the net effect of AMPK activation is stimulation of hepatic fatty acid oxidation, ketogenesis, stimulation of skeletal muscle fatty acid oxidation and glucose uptake, inhibition of cholesterol synthesis, lipogenesis, and triglyceride synthesis, inhibition of adipocyte lipogenesis, inhibition of adipocyte lipolysis, and modulation of insulin secretion by pancreatic beta-cells.

S6K is a downstream target of mTOR signaling.

### Methods and Compositions to Overexpress, Activate or Increase Targeted Proteins

As shown herein the overexpression, activation and/or increase of certain proteins including but not limited to UNC-51-like kinase (ULK1), microtubule-associated protein light chain 3A (LC3), and adenosine monophosphate protein kinase (AMPK) can mimic the effect of time-restricted feeding and increase and/or extend lifespan and/or healthspan and/or delay aging.

Thus, one embodiment of the present disclosure is a method of increasing and/or extending lifespan and/or healthspan in a subject by administering a therapeutically effective amount of an agent which mimics the effects of time-restricted feeding by overexpressing, activating or increasing ULK1, LC3, and/or AMPK.

A further embodiment of the present disclosure is a method of delaying aging in a subject by administering a therapeutically effective amount of an agent which mimics the effects of time-restricted feeding by overexpressing, activating or increasing ULK1, LC3, and/or AMPK.

Agents that can be used in these methods include but are not limited to agents for increasing the expression of the gene encoding ULK1, LC3, and/or AMPK, and include nucleic acids which encode the ULK1, LC3, and/or AMPK protein, or the entire ULK1, LC3, and/or AMPK gene, or a nucleic acid that is substantially homologous to the ULK1, LC3, and/or AMPK gene, or a variant, mutant, fragment, homologue or derivative of the ULK1, LC3, and/or AMPK gene that produces a protein that maintains or increases its function.

In some embodiments, the present disclosure provides methods of administering to a subject in need thereof a therapeutically effective amount of a composition, or compositions or a viral vector, comprising a nucleic acid encoding ULK1, LC3, and/or AMPK.

Any suitable viral system could be utilized including AAV, lentiviral vectors, or other suitable vectors.

Nucleic acid sequences of transgenes described herein may be designed based on the knowledge of the specific composition (e.g., viral vector) that will express the transgene. For example, one type of transgene sequence includes a reporter sequence, which upon expression produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

In some embodiments of the current disclosure the transgenes would encode a functional protein including but not limited to ULK1, LC3, and/or AMPK.

By “ULK1”, “ULK1”, Ulk1”, and “Ulk1” is meant to include the DNA, RNA, mRNA, cDNA, recombinant DNA or RNA, or the protein arising from the ULK1 gene.

It is noted that as used herein ULK1 can refer to the gene or the protein encoded for by the gene, as appropriate in the specific context utilized. Additionally, in certain contexts, the reference will be to the mouse gene or protein, and in others the human gene or protein as appropriate in the specific context.

The human ULK1 gene (GenBank: 8404) can be used to obtain a transgene.

In some embodiments, the transgene encodes ULK1. The ULK1 may have an amino acid sequence that is at least 85% identical to the amino acid sequence of human ULK1 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of human ULK1). In some embodiments, the ULK1 has an amino acid sequence that is at least 90% identical to the amino acid sequence of human ULK1 (e.g., an amino acid sequence that is 90%, 91%, 92%, 93%, 94%, 95%, 96%, 97%, 98%, 99%, or 100% identical to the amino acid sequence of human ULK1). In some embodiments, the ULK1 has an amino acid sequence that is at least 95% identical to the amino acid sequence of human ULK1 (e.g., an amino acid sequence that is 95%, 96%, 97%, 98%, 99%, or 100% identical to the amino acid sequence of human ULK1).

In some embodiments, the ULK1 has an amino acid sequence that differs from human ULK1 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the ULK1 has an amino acid sequence that differs from human ULK1 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding ULK1 has a nucleic acid sequence that is at least 70% identical to the nucleic acid sequence of human ULK1 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of human ULK1). In some embodiments, the transgene encoding ULK1 has a nucleic acid sequence that is at least 85% identical to the nucleic acid sequence of human ULK1 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of human ULK1). In some embodiments, the transgene encoding ULK1 has a nucleic acid sequence that is at least 90% identical to the nucleic acid sequence of human ULK1 (e.g., a nucleic acid sequence that is 90%, 91%, 92%, 93%, 94%, 95%, 96%, 97%, 98%, 99%, or 100% identical to the nucleic acid sequence of human ULK1). In some embodiments, the transgene encoding ULK1 has a nucleic acid sequence that is at least 95% identical to the nucleic acid sequence of human ULK1 (e.g., a nucleic acid sequence that is 95%, 96%, 97%, 98%, 99%, or 100% identical to the nucleic acid sequence of human ULK1).

By “LC3”, “LC3”, LC3”, and “LC3” is meant to include the DNA, RNA, mRNA, cDNA, recombinant DNA or RNA, or the protein arising from the LC3 gene.

It is noted that as used herein LC3 can refer to the gene or the protein encoded for by the gene, as appropriate in the specific context utilized. Additionally, in certain contexts, the reference will be to the mouse gene or protein, and in others the human gene or protein as appropriate in the specific context.

The human LC3 gene (GenBank: 84557 and 81631) can be used to obtain a transgene.

In some embodiments, the transgene encodes LC3. The LC3 may have an amino acid sequence that is at least 85% identical to the amino acid sequence of human LC3 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of human LC3). In some embodiments, the LC3 has an amino acid sequence that is at least 90% identical to the amino acid sequence of human LC3 (e.g., an amino acid sequence that is 90%, 91%, 92%, 93%, 94%, 95%, 96%, 97%, 98%, 99%, or 100% identical to the amino acid sequence of human LC3). In some embodiments, the LC3 has an amino acid sequence that is at least 95% identical to the amino acid sequence of human LC3 (e.g., an amino acid sequence that is 95%, 96%, 97%, 98%, 99%, or 100% identical to the amino acid sequence of human LC3).

In some embodiments, the LC3 has an amino acid sequence that differs from human LC3 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the LC3 has an amino acid sequence that differs from human LC3 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 21, 22, 23, 24, 25, or more, conservative amino acid substitutions).

In some embodiments, the transgene encoding LC3 has a nucleic acid sequence that is at least 70% identical to the nucleic acid sequence of human LC3 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of human LC3). In some embodiments, the transgene encoding LC3 has a nucleic acid sequence that is at least 85% identical to the nucleic acid sequence of human LC3 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of human LC3). In some embodiments, the transgene encoding LC3 has a nucleic acid sequence that is at least 90% identical to the nucleic acid sequence of human LC3 (e.g., a nucleic acid sequence that is 90%, 91%, 92%, 93%, 94%, 95%, 96%, 97%, 98%, 99%, or 100% identical to the nucleic acid sequence of human LC3). In some embodiments, the transgene encoding LC3 has a nucleic acid sequence that is at least 95% identical to the nucleic acid sequence of human LC3 (e.g., a nucleic acid sequence that is 95%, 96%, 97%, 98%, 99%, or 100% identical to the nucleic acid sequence of human LC3).

By “AMPK”, “AMPK”, AMPK″, and “AMPK” is meant to include the DNA, RNA, mRNA, cDNA, recombinant DNA or RNA, or the protein arising from the AMPK gene.

It is noted that as used herein AMPK can refer to the gene or the protein encoded for by the gene, as appropriate in the specific context utilized. Additionally, in certain contexts, the reference will be to the mouse gene or protein, and in others the human gene or protein as appropriate in the specific context.

The human AMPK gene (GenBank: 5562) can be used to obtain a transgene.

In some embodiments, the transgene encodes AMPK. The AMPK may have an amino acid sequence that is at least 85% identical to the amino acid sequence of human AMPK (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of human AMPK). In some embodiments, the AMPK has an amino acid sequence that is at least 90% identical to the amino acid sequence of human AMPK (e.g., an amino acid sequence that is 90%, 91%, 92%, 93%, 94%, 95%, 96%, 97%, 98%, 99%, or 100% identical to the amino acid sequence of human AMPK). In some embodiments, the AMPK has an amino acid sequence that is at least 95% identical to the amino acid sequence of human AMPK (e.g., an amino acid sequence that is 95%, 96%, 97%, 98%, 99%, or 100% identical to the amino acid sequence of human AMPK).

In some embodiments, the AMPK has an amino acid sequence that differs from human AMPK by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the AMPK has an amino acid sequence that differs from human AMPK by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 16, 17, 18, 19, 20, 21, 22, 23, 24, 25, or more, conservative amino acid substitutions).

In some embodiments, the transgene encoding AMPK has a nucleic acid sequence that is at least 70% identical to the nucleic acid sequence of human AMPK (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of human AMPK). In some embodiments, the transgene encoding AMPK has a nucleic acid sequence that is at least 85% identical to the nucleic acid sequence of human AMPK (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of human AMPK). In some embodiments, the transgene encoding AMPK has a nucleic acid sequence that is at least 90% identical to the nucleic acid sequence of human AMPK (e.g., a nucleic acid sequence that is 90%, 91%, 92%, 93%, 94%, 95%, 96%, 97%, 98%, 99%, or 100% identical to the nucleic acid sequence of human AMPK). In some embodiments, the transgene encoding AMPK has a nucleic acid sequence that is at least 95% identical to the nucleic acid sequence of human AMPK (e.g., a nucleic acid sequence that is 95%, 96%, 97%, 98%, 99%, or 100% identical to the nucleic acid sequence of human AMPK).

In some embodiments, the transgene encoding ULK1, LC3, and/or AMPK is codon optimized to increase efficiency.

Codon optimization tools are known in the art.

Alternatively, administering the proteins can be used in the methods. This includes the administration of a polypeptide, or a variant thereof having at least 80% sequence identity with the ULK1, LC3, and/or AMPK polypeptides.

In an embodiment, the variant of the polypeptide has at least 81% sequence identity with the sequence of the polypeptide of which it is a variant. Thus, preferably, the variant of the polypeptide has at least 81%, 82%, 83%, 84%, 85%, 86%, 87%, 88%, 89%, 90%, 91%, 92%, 93%, 94%, 95%, 96%, 97%, 98% or 99% sequence identity with the sequence of the ULK1, LC3, and/or AMPK polypeptide. Such variants may be made, for example, using the methods of recombinant DNA technology, protein engineering and site-directed mutagenesis, which are well known in the art, and discussed in more detail below.

The percent sequence identity between two polypeptides may be determined using suitable computer programs.

Biologically active fragments (also referred to as biologically active peptides) or variants include any fragments or variants of a protein that retain an activity of the protein.

Polypeptides may be prepared using an in vivo or in vitro expression system. Preferably, an expression system is used that provides the polypeptides in a form that is suitable for pharmaceutical use, and such expression systems are known to the skilled person. As is clear to the skilled person, polypeptides of the invention suitable for pharmaceutical use can be prepared using techniques for peptide synthesis.

The polypeptide or variants thereof, may be made by chemical synthesis, again using methods well known in the art for many years. In certain embodiments, polypeptides for administration to a patient may be in the form of a fusion molecule in which the polypeptide is attached to a fusion partner to form a fusion protein. Many different types of fusion partners are known in the art. One skilled in the art can select a suitable fusion partner according to the intended use of the fusion protein. Examples of fusion partners include polymers, polypeptides, lipophilic moieties, and succinyl groups. Certain useful protein fusion partners include serum albumin and an antibody Fc domain, and certain useful polymer fusion partners include, but are not limited to, polyethylene glycol, including polyethylene glycols having branched and/or linear chains. In certain embodiments, the polypeptide may be PEGylated, or may comprise a fusion protein with an Fc fragment.

In an embodiment, the polypeptide may be fused to or may comprise additional amino acids in a sequence that facilitates entry into cells (i.e., a cell-penetrating peptide). Thus, for example, the protein or variant thereof or a polypeptide may further comprise the sequence of a cell-penetrating peptide (also known as a protein transduction domain) that facilitates entry into cells. As is well known in the art, cell-penetrating peptides are generally short peptides of up to 30 residues having a net positive charge and act in a receptor-independent and energy-independent manner.

Additionally or alternatively, the polypeptide may be fused to or may comprise additional amino acids in a sequence that facilitates entry into the nucleus (i.e., a nuclear localization sequence (NLS), aka nuclear localization domain (NLD)). Thus, for example, the protein or variant thereof may further comprise the sequence of an NLS that facilitates entry into the nucleus. NLS includes any polypeptide sequence that, when fused to a target polypeptide, is capable of targeting it to the nucleus. Typically, the NLS is one that is not under any external regulation (e.g., calcineurin regulation) but which permanently translocates a target polypeptide to the nucleus.

It is appreciated that the sequence of the cell-penetrating peptide and/or the NLS may be adjacent to the sequence of the protein or variant, or these sequences may be separated by one or more amino acids residues, such as glycine residues, acting as a spacer.

Therapeutic proteins produced as an Fc-chimera are known in the art. For example, Etanercept, the extracellular domain of TNFR2 combined with an Fc fragment, is a therapeutic polypeptide used to treat autoimmune diseases, such as rheumatoid arthritis.

Other protein modifications to stabilize a polypeptide, for example to prevent degradation, as are well known in the art may also be employed. Specific amino acids may be modified to reduce cleavage of the polypeptide in vivo. Typically, N- or C-terminal regions are modified to reduce protease activity on the polypeptide. A stabilizing modification is any modification capable of stabilizing a protein, enhancing the in vitro half-life of a protein, enhancing circulatory half-life of a protein and/or reducing proteolytic degradation of a protein. For example, polypeptides may be linked to the serum albumin or a derivative of albumin. Methods for linking polypeptides to albumin or albumin derivatives are well known in the art.

The fusion partner may be attached, either covalently or non-covalently, to the amino-terminus or the carboxy-terminus of the polypeptide. The attachment may also occur at a location within the polypeptide other than the amino-terminus or the carboxy-terminus, for example, through an amino acid side chain (such as, for example, the side chain of cysteine, lysine, histidine, serine, or threonine).

Pharmaceutical or small molecule agents which target ULK1, LC3, and/or AMPK can also be used in the methods provided herein. These agents include but are not limited to AICAR, Metformin HC1, LYN-1604, BL-918, and rac-BL-918.

AICAR is an AMPK activator known to induce autophagy and has been used for cardiac indications. AICAR is otherwise known as 5-Aminoimidazole-4-carboxamide 1-β-D-ribofuranoside, N1-(β-D-Ribofuranosyl)-5-aminoimidazole-4-carboxamide or Acadesine, and has the following formula: C₉H₁₄N₄O₅.

Metformin HCl is also an AMPK activator. Metformin HCl is also known as 1,1-Dimethylbiguanide hydrochloride or Metformin and has the following formula: NH₂C(═NH)NHC(═NH)N(CH₃)₂·HCl. Metformin is an oral type 2 diabetes pharmaceutical sold under the trade names Fortamet, Glucophage, Glucophage XR, Glumetza, and Riomet

ULK1 activators include LYN-1604 hydrochloride and LYN-1604 dihydrochloride which have the following chemical formula and are currently being used for research for triple negative breast cancer:

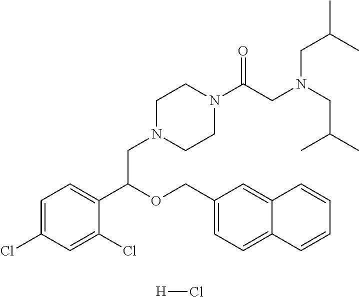 [SMILES: CC(C)CN(CC(=O)N1CCN(CC(OCc2ccc3ccccc3c2)c2ccc(Cl)cc2Cl)CC1)CC(C)C ; Cl]

Other ULK1 activators include BL-918 which is an orally active ULK1 activator with an EC₅₀ of 24.14 nM. BL-918 may potentially be used for Parkinson's disease (PD) treatment. BL-918 has the following chemical formula:

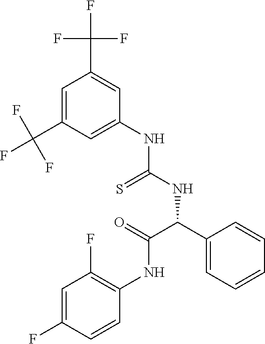 [SMILES: O=C(Nc1ccc(F)cc1F)[C@H](NC(=S)Nc1cc(C(F)(F)F)cc(C(F)(F)F)c1)c1ccccc1]

Rac-BL-918 is the racemate of BL-918, with the same activity.

### Methods and Compositions to Inhibit or Decrease Targeted Proteins

As shown herein the inhibition of certain proteins including but not limited to ribosomal protein S6 kinase beta-1 (S6K) can mimic the effect of time-restricted feeding and increase and/or extend lifespan and/or healthspan and/or delay aging.

Thus, one embodiment of the present disclosure is a method of increasing and/or extending lifespan and/or healthspan in a subject by administering a therapeutically effective amount of an agent which mimics the effects of time-restricted feeding by inhibiting or decreasing S6K.

A further embodiment of the present disclosure is a method of delaying aging in a subject by administering a therapeutically effective amount of an agent which mimics the effects of time-restricted feeding by inhibiting or decreasing S6K.

By “S6K,” “S6K,” “S6K,” “S6K” is meant to include the DNA, RNA, mRNA, cDNA, recombinant DNA or RNA, or the protein arising from the S6K gene or S6K interactors. The human reference sequence can be found at GenBank 6198.

It is noted that as used herein S6K can refer to the gene or the protein encoded for by the gene, as appropriate in the specific context utilized. Additionally, in certain contexts, the reference will be to the mouse gene or protein, and in others the human gene or protein as appropriate in the specific context.

Any isoform of any S6K may be inhibited by the present inhibitors. The present inhibitors may target the wild-type or mutant form of S6K.

As used herein, the term “inhibitor” refers to agents capable of down-regulating or otherwise decreasing or suppressing the amount/level and/or activity of S6K.

The mechanism of inhibition may be at the genetic level (e.g., interference with or inhibit expression, transcription or translation) or at the protein level (e.g., binding, competition).

A wide variety of suitable inhibitors may be employed, guided by art-recognized criteria such as efficacy, toxicity, stability, specificity, and half-life.

Any suitable viral knockdown system could be utilized for decreasing S6K or S6K mRNA levels including AAV, lentiviral vectors, or other suitable vectors.

Additionally, specifically targeted delivery of S6K blocking molecule (nucleic acid, peptide, or small molecule) could be delivered by targeted liposome, nanoparticle or other suitable means.

Endonucleases

Methods for modification of genomic DNA are well known in the art. For example, methods may use a DNA digesting agent to modify the DNA by either the non-homologous end joining DNA repair pathway (NHEJ) or the homology directed repair (HDR) pathway. The term “DNA digesting agent” refers to an agent that is capable of cleaving bonds (i.e., phosphodiester bonds) between the nucleotide subunits of nucleic acids.

In one embodiment, the DNA digesting agent is a nuclease. Nucleases are enzymes that hydrolyze nucleic acids. Nucleases may be classified as endonucleases or exonucleases. An endonuclease is any of a group of enzymes that catalyze the hydrolysis of bonds between nucleic acids in the interior of a DNA or RNA molecule. An exonuclease is any of a group of enzymes that catalyze the hydrolysis of single nucleotides from the end of a DNA or RNA chain. Nucleases may also be classified based on whether they specifically digest DNA or RNA. A nuclease that specifically catalyzes the hydrolysis of DNA may be referred to as a deoxyribonuclease or DNase, whereas a nuclease that specifically catalyzes the hydrolysis of RNA may be referred to as a ribonuclease or an RNase. Some nucleases are specific to either single-stranded or double-stranded nucleic acid sequences. Some enzymes have both exonuclease and endonuclease properties. In addition, some enzymes are able to digest both DNA and RNA sequences.

S6K may be inhibited by using a sequence-specific endonuclease that target the gene encoding S6K.

Non-limiting examples of the endonucleases include a zinc finger nuclease (ZFN), a ZFN dimer, a ZFNickase, a transcription activator-like effector nuclease (TALEN), or a RNA-guided DNA endonuclease (e.g., CRISPR/Cas). Meganucleases are endonucleases characterized by their capacity to recognize and cut large DNA sequences (12 base pairs or greater). Any suitable meganuclease may be used in the present methods to create double-strand breaks in the host genome, including endonucleases in the LAGLIDADG and PI-Sce family.

One aspect of the present disclosure provides RNA-guided endonucleases. RNA-guided endonucleases also comprise at least one nuclease domain and at least one domain that interacts with a guide RNA. An RNA-guided endonuclease is directed to a specific nucleic acid sequence (or target site) by a guide RNA. The guide RNA interacts with the RNA-guided endonuclease as well as the target site such that, once directed to the target site, the RNA-guided endonuclease is able to introduce a double-stranded break into the target site nucleic acid sequence. Since the guide RNA provides the specificity for the targeted cleavage, the endonuclease of the RNA-guided endonuclease is universal and can be used with different guide RNAs to cleave different target nucleic acid sequences.

One example of an RNA guided sequence-specific nuclease system that can be used with the methods and compositions described herein includes the CRISPR system (Wiedenheft, et al. 2012 Nature 482:331-338; Jinek, et al. 2012 Science 337:816-821; Mali, et al. 2013 Science 339:823-826; Cong, et al. 2013. Science 339:819-823). The CRISPR (Clustered Regularly Interspaced Short Palindromic Repeats) system exploits RNA-guided DNA-binding and sequence-specific cleavage of target DNA. The guide RNA/Cas combination confers site specificity to the nuclease. A single guide RNA (sgRNA) contains about 20 nucleotides that are complementary to a target genomic DNA sequence upstream of a genomic PAM (protospacer adjacent motifs) site (e.g., NGG) and a constant RNA scaffold region. The Cas (CRISPR-associated) protein binds to the sgRNA and the target DNA to which the sgRNA binds and introduces a double-strand break in a defined location upstream of the PAM site. Cas9 harbors two independent nuclease domains homologous to HNH and RuvC endonucleases, and by mutating either of the two domains, the Cas9 protein can be converted to a nickase that introduces single-strand breaks (Cong, et al. 2013 Science 339:819-823). It is specifically contemplated that the methods and compositions of the present disclosure can be used with the single- or double-strand-inducing version of Cas9, as well as with other RNA-guided DNA nucleases, such as other bacterial Cas9-like systems. The sequence-specific nuclease of the present methods and compositions described herein can be engineered, chimeric, or isolated from an organism. The nuclease can be introduced into the cell in form of a DNA, mRNA and protein.

In one embodiment, the methods of the present disclosure comprise using one or more sgRNAs to target and/or remove or suppress S6K.

Inhibitory Nucleic Acids That Hybridize to or Target S6K

In certain embodiments, the S6K inhibitor used in the present methods and compositions is a polynucleotide that reduces or inhibits expression of S6K. Thus, the method involves administering an effective amount of a polynucleotide that specifically targets nucleotide sequence(s) encoding S6K. The polynucleotides reduce expression of S6K, to yield reduced levels of the gene product (the translated polypeptide).

The nucleic acid target of the polynucleotides (e.g., antisense oligonucleotides, and ribozymes) may be any location within the gene or transcript of S6K.

In further embodiments, the S6K inhibitor used in the present methods and compositions is a polynucleotide that reduces expression of S6K. Thus, the method involves administering an effective amount of a polynucleotide that specifically targets nucleotide sequence(s) encoding S6K. The polynucleotides reduce expression of S6K, to yield reduced levels of the gene product (the translated polypeptide).

The nucleic acid target of the polynucleotides (e.g., antisense oligonucleotides, and ribozymes) may be any location within the gene or transcript of S6K.

The inhibitory nucleic acids may be RNA interference or RNAi, an antisense RNA, a ribozyme, or combinations thereof.

“RNA interference”, or “RNAi” is a form of post-transcriptional gene silencing (“PTGS”), and comprises the introduction of, e.g., double-stranded RNA into cells. The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. RNAi can work in human cells if the RNA strands are provided as pre-sized duplexes of about 19 nucleotide pairs, and RNAi worked particularly well with small unpaired 3′ extensions on the end of each strand (Elbashir et al. Nature 411:494-498 (2001)).

The inhibitory nucleic acid may be a short RNA molecule, such as a short interfering RNA (siRNA), a small temporal RNA (stRNA), a short hairpin RNA (shRNA), and a micro-RNA (miRNA). Short interfering RNAs silence genes through an mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science, 305(5688):1289-92 (2004); He and Hannon, Nat Rev Genet. 5(7):522-31 (2004).

Software programs for predicting siRNA sequences to inhibit the expression of a target protein are commercially available and find use. One program, siDESIGN from Dharmacon, Inc. (Lafayette, Colo.), permits predicting siRNAs for any nucleic acid sequence, and is available on the internet at dharmacon.com. Programs for designing siRNAs are also available from others, including Genscript (available on the internet at genscript.com/ssl-bin/app/rnai) and, to academic and non-profit researchers, from the Whitehead Institute for Biomedical Research found on the worldwide web at “jura.wi.mit.edu/pubint/http://iona.wi.mit.edu/siRNAext/.”

The inhibitory nucleic acids may be an antisense nucleic acid sequence that is complementary to a target region within the mRNA of S6K or S6K. The antisense polynucleotide may bind to the target region and inhibit translation. The antisense oligonucleotide may be DNA or RNA or comprise synthetic analogs of ribo-deoxynucleotides. Thus, the antisense oligonucleotide inhibits expression of S6K.

An antisense oligonucleotide can be, for example, about 7, 10, 15, 20, 25, 30, 35, 40, 50, 55, 60, 65, 70, 75, 80, or more nucleotides in length.

The antisense nucleic acid molecules of the invention may be administered to a subject or generated in situ such that they hybridize with or bind to the mRNA of S6K.

Ribozyme

The inhibitor may be a ribozyme that inhibits expression of the S6K or S6K gene.

Ribozymes can be chemically synthesized and structurally modified to increase their stability and catalytic activity using methods known in the art. Ribozyme encoding nucleotide sequences can be introduced into host cells through gene-delivery mechanisms known in the art.

Antibodies

The present inhibitors can be an antibody or antigen-binding portion thereof that is specific to S6K.

The antibody or antigen-binding portion thereof may be the following: (a) a whole immunoglobulin molecule; (b) an scFv; (c) a Fab fragment; (d) an F(ab')2; and (e) a disulfide linked Fv. The antibody or antigen-binding portion thereof may be monoclonal, polyclonal, chimeric and humanized The antibodies may be murine, rabbit or human/humanized antibodies.

Pharmaceuticals or Small Molecule Inhibitors

Pharmaceutical or small molecule agents which target S6K can also be used in the methods provided herein. These agents include but are not limited to rapamycin. Rapamycin is derived from Streptomyces hygroscopicus. It is also known as 23,27-Epoxy-3H-pyrido[2,1-c][1,4]oxaazacyclohentriacontine, Sirolimus, or AY 22989, and has the formula:

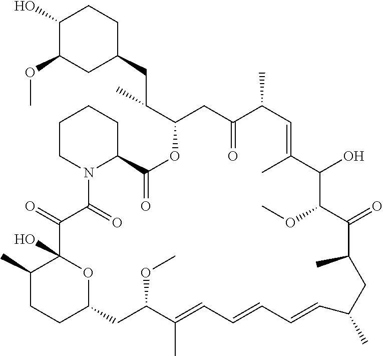 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)C(O)[C@@H](OC)C(=O)[C@H](C)C[C@H](C)/C=C/C=C/C=C/1C]

Rapamycin is used to treat organ transplant failure, vascular malformations and lymphangioleiomyomatosis (LAM), a rare, progressive lung disease.

Vectors

In some embodiments of the disclosure, the nucleic acids for use in the disclosed methods are contained in a vector, such as a viral vector. In some embodiments of the disclosure, the composition for use in the disclosed methods comprises a vector, such as a viral vector. The viral vector may be, for example, an AAV, adenovirus, lentivirus, retrovirus, poxvirus, baculovirus, herpes simplex virus, vaccinia virus, or a synthetic virus (e.g., a chimeric virus, mosaic virus, or pseudotyped virus, and/or a virus that contains a foreign protein, synthetic polymer, nanoparticle, or small molecule).

The vector may also include conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide.

In some embodiments of the disclosure, the transgenes are operably linked to promoters that induce expression of the transgenes in the proper cells. Alternatively, ubiquitous promoters may be used including without limitation, CMV, EF1, CAG, CB7, PGK and SFFV.

Other regulatory elements may also be used such as a polyadenylation sequence and post-transcriptional regulatory elements, for efficient pre-mRNA processing and increasing gene expression, respectively. For nucleic acids encoding proteins, a polyadenylation sequence generally is inserted following the transgene sequences. Examples of polyadenylation sequences include SV40, bGHpolyA and spA. Examples of post-transcriptional regulatory elements include WPRE, WPRE3 and HPRE.

In some embodiments, optimized combinations of polyadenylation sequences and post-transcriptional regulatory elements may be used in the vectors.

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors may optionally include 5′ leader or signal sequences.

In some embodiments, the regulatory sequences impart tissue-specific gene expression capabilities. In some cases, the tissue-specific regulatory sequences bind tissue-specific transcription factors that induce transcription in a tissue specific manner.

### Pharmaceutical Compositions and Methods of Administration

The present methods include the administration of agents which mimic the effects of time-restricted feeding, which in some embodiments is a pharmaceutical or a small molecule. Preferred methods of administration include oral; mucosal, such as nasal, sublingual, vaginal, buccal, or rectal; parenteral, such as subcutaneous, intravenous, bolus injection, intramuscular, or intra-arterial; or transdermal administration to a subject. Thus, the agent must be in the appropriate form for administration of choice.

When the agent is a nucleic acid such as DNA, RNA, interfering RNA or microRNA, methods for delivery include receptor mediated endocytosis where the nucleic acid is coupled to a targeting molecule that can bind to a specific cell surface receptor, inducing endocytosis and transfer of the nucleic acid into cells. Coupling is normally achieved by covalently linking poly-lysine to the receptor molecule and then arranging for (reversible) binding of the negatively charged nucleic acid to the positively charged poly-lysine component. Another approach utilizes the transferrin receptor or folate receptor which is expressed in many cell types. When producing the microRNA for this method of administration, the microRNA could be manufactured to have a guide strand which is identical to the microRNA of interest and a passenger strand that is modified and linked to a molecule for increasing cellular uptake.

Liposomes are spherical vesicles composed of synthetic lipid bilayers which mimic the structure of biological membranes. The nucleic acid to be transferred is packaged in vitro with the liposomes and used directly for transferring the nucleic acid to a suitable target tissue in vivo. The lipid coating allows the nucleic acid to survive in vivo, bind to cells and be endocytosed into the cells. Cationic liposomes (where the positive charge on liposomes stabilize binding of negatively charged DNA), have are one type of liposome.

The nucleic acids can also be administered with a lipid to increase cellular uptake, The nucleic acids may be administered in combination with a cationic lipid, including but not limited to, lipofectin, DOTMA, DOPE, and DOTAP.

Other lipid or liposomal formulations including nanoparticles and methods of administration have been described as for example in U.S. Patent Publication 2003/0203865, 2002/0150626, 2003/0032615, and 2004/0048787. Methods used for forming particles are also disclosed in U.S. Pat. Nos. 5,844,107, 5,877,302, 6,008,336, 6,077,835, 5,972,901, 6,200,801, and 5,972,900.

Another method for delivery of nucleic acid to the proper tissue or cell is by using viral vector, such as an adeno-associated viruses (AAV). Nucleic acid delivered in these viral vectors is continually expressed, replacing the expression of the nucleic acid that is not expressed in the subject. Also, AAV have different serotypes allowing for tissue-specific delivery due to the natural tropism toward different organs of each individual AAV serotype as well as the different cellular receptors with which each AAV serotype interacts. The use of tissue-specific promoters for expression allows for further specificity in addition to the AAV serotype.

Other mammalian virus vectors that can be used to deliver the RNA include oncoretroviral vectors, adenovirus vectors, Herpes simplex virus vectors, and lentiviruses.

The viral vectors may be delivered to a subject in compositions according to any appropriate methods known in the art. The viral vector, preferably suspended in a physiologically compatible carrier (e.g., in a composition), may be administered to a subject. In certain embodiments, compositions may comprise a vector alone, or in combination with one or more other viruses (e.g., a viral vector encoding having one or more different transgenes or an endonuclease).

Such compositions for administration may comprise a therapeutically effective amount of the active agent and a pharmaceutically acceptable carrier. The phrase “pharmaceutically acceptable” refers to molecular entities and compositions that are physiologically tolerable and do not typically produce an allergic or similar untoward reaction, such as gastric upset, dizziness and the like, when administered to a human, and approved by a regulatory agency of the Federal or a state government or listed in the U.S. Pharmacopeia or other generally recognized pharmacopeia for use in animals, and more particularly in humans “Carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, pectin, peanut oil, sesame oil and the like. The composition, if desired, can also contain minor amounts of wetting or emulsifying agents, or pH buffering agents.

The selection of the carrier is not a limitation of the present invention.

Optionally, the compositions may contain, in addition to the agent and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

These compositions can take the form of solutions, suspensions, emulsions, tablets, pills, capsules, powders, sustained-release formulations, cachets, troches, lozenges, dispersions, suppositories, ointments, cataplasms (poultices), pastes, powders, dressings, creams, plasters, patches, aerosols, gels, liquid dosage forms suitable for parenteral administration to a patient, and sterile solids (e.g., crystalline or amorphous solids) that can be reconstituted to provide liquid dosage forms suitable for parenteral administration to a patient. Such compositions will contain a therapeutically effective amount of the agent or compound, preferably in purified form, together with a suitable form of carrier so as to provide the form for proper administration to the patient. The formulation should suit the mode of administration.

Pharmaceutical compositions adapted for oral administration may be capsules, tablets, powders, granules, solutions, syrups, suspensions (in non-aqueous or aqueous liquids), or emulsions. Tablets or hard gelatin capsules may comprise lactose, starch or derivatives thereof, magnesium stearate, sodium saccharine, cellulose, magnesium carbonate, stearic acid or salts thereof. Soft gelatin capsules may comprise vegetable oils, waxes, fats, semi-solid, or liquid polyols. Solutions and syrups may comprise water, polyols, and sugars. An active agent intended for oral administration may be coated with or admixed with a material that delays disintegration and/or absorption of the active agent in the gastrointestinal tract. Thus, the sustained release may be achieved over many hours and if necessary, the active agent can be protected from degradation within the stomach. Pharmaceutical compositions for oral administration may be formulated to facilitate release of an active agent at a particular gastrointestinal location due to specific pH or enzymatic conditions.

Pharmaceutical compositions adapted for transdermal administration may be provided as discrete patches intended to remain in intimate contact with the epidermis of the recipient over a prolonged period of time.

Pharmaceutical compositions adapted for nasal and pulmonary administration may comprise solid carriers such as powders which can be administered by rapid inhalation through the nose. Compositions for nasal administration may comprise liquid carriers, such as sprays or drops. Alternatively, inhalation directly through into the lungs may be accomplished by inhalation deeply or installation through a mouthpiece. These compositions may comprise aqueous or oil solutions of the active ingredient. Compositions for inhalation may be supplied in specially adapted devices including, but not limited to, pressurized aerosols, nebulizers or insufflators, which can be constructed so as to provide predetermined dosages of the active ingredient.

Pharmaceutical compositions adapted for rectal administration may be provided as suppositories or enemas. Pharmaceutical compositions adapted for vaginal administration may be provided as pessaries, tampons, creams, gels, pastes, foams or spray formulations.

Pharmaceutical compositions adapted for parenteral administration include aqueous and non-aqueous sterile injectable solutions or suspensions, which may contain anti-oxidants, buffers, bacteriostats, and solutes that render the compositions substantially isotonic with the blood of the subject. Other components which may be present in such compositions include water, alcohols, polyols, glycerine, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the host. The person responsible for administration will, in any event, determine the appropriate dose for the individual host.

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active ingredient or more, although the percentage of the active ingredient(s) may, of course, be varied and may conveniently be between about 1 or 2% and about 70% or 80% or more of the weight or volume of the total formulation. Naturally, the amount of active ingredient in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Selection of a therapeutically effective dose will be determined by the skilled artisan considering several factors which will be known to one of ordinary skill in the art. Such factors include the particular form of the agent, and its pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

Doses can be adjusted to optimize the effects in the subject. Additionally, a subject can be monitored for improvement of their condition prior to increasing the dosage.

In some embodiments, the administration of the agent is done at night.

### Kits

Also within the scope of the present disclosure are kits for practicing the disclosed methods.

In some embodiments, the kit can comprise instructions for use in any of the methods described herein. The included instructions can comprise a description of administration of the agents to a subject to achieve the intended activity in a subject. The kit may further comprise a description of selecting a subject suitable for treatment based on identifying whether the subject is in need of the treatment.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, and proper storage conditions,

### EXAMPLES

The present invention may be better understood by reference to the following non-limiting examples, which are presented in order to more fully illustrate the preferred embodiments of the invention. They should in no way be construed to limit the broad scope of the invention.

### Example 1—Materials and Methods For Examples 2-7

### Fly Strains

w¹¹¹⁸ Canton-S (CS) were used as a “wild-type” strain for determining optimal feeding regimens. UAS-DN-S6K (6911), UAS-CA-S6K (6914), UAS-DN-AMPK (32112), UAS-CA-AMPK (32110) UAS-atg1-RNAi (44034), UAS-atg8-RNAi (34340), and UAS-atg1 (51654) were obtained from the Bloomington Stock Center. tubulin-GAL4 from John Carlson, UAS-GFP-Atg8 was from Eric Baehrecke, and daughterless-Gene-Switch (daGS) from David W. Walker. period (per01) mutants, timeless-GAL4 and period-GAL4 were obtained from Jaga Giebultowicz, which were newly outcrossed for 12 generations to a w¹¹¹⁸ 535 Canton-S (CS) control. UAS-gRNA, UAS-CAS9 lines were those utilized as previously described (Delventhal et al. 2019; Ulgherait et al. 2020). Previously outcrossed cycle (cyc⁰¹) mutants were obtained from Sheyum Syed with a CS control strain. All experiments with multiple transgenes used flies that have undergone 12 generations of out-crossing into a w¹¹¹⁸ Canton-S (CS) control and/or per01, w¹¹¹⁸ Canton-S (CS) mutant background.

### Fly Media

Developmental media consisted of standard yeast-cornmeal-agar media (Archon Scientific, Glucose recipe: 7.6% w/v glucose, 3.8% w/v yeast, 5.3% w/v cornmeal, w/v 0.6% agar, 0.5% v/v propionic acid, 0.1% w/v methyl paraben, 0.3% v/v ethanol). Adult flies that eclosed within a 24-hour period were collected and transferred to “adult medium” containing 4% glucose, 2% sucrose, 8% cornmeal, 1% agar, and either 0%, 0.5%, 1%, 2%, 3%, 6%, or 10% yeast extract (Difco) supplemented with 1.5% methylparaben mix (10% methylparaben dissolved in ethanol) and 1% propionic acid for lifespan and biochemical analysis. Standard adult medium contained 3% yeast extract. All percentages given in w/v except methylparaben mix, propionic acid given in v/v. Fasting media consisted of 1% agar in ddH₂O made fresh daily.

### Drug Supplementation in Media For Lifespan

All drugs were supplemented into cooled (65° C.) liquid adult medium (containing 3% yeast extract) following preparation to the following final concentration(s): RU486 (Cayman Chemical) was dissolved in ethanol was supplemented into medium after day 5 of eclosion at a final concentration of 100 μg/mL (with the exception of daGS>UAS-atg1 and controls, 0.5 μg/mL) vehicle controls were supplemented with same volume of ethanol alone. Timed RU486 feeding of daGS>UAS-atg1 consisted of flies being fed 0.5 μg/mL in standard adult media or fasting media only during the fasting phase, then flipped to vehicle control food the following morning only from days 10-40 of adulthood. Vehicle only controls were flipped at the same time for consistency. For gut microbe clearance, the medium contained 500 μg/mL ampicillin, 50 μg/mL tetracycline, and 200 μg/mL rifamycin in 50% ethanol. Same volume of 50% ethanol alone was used as the vehicle control.

### Lifespan Analysis

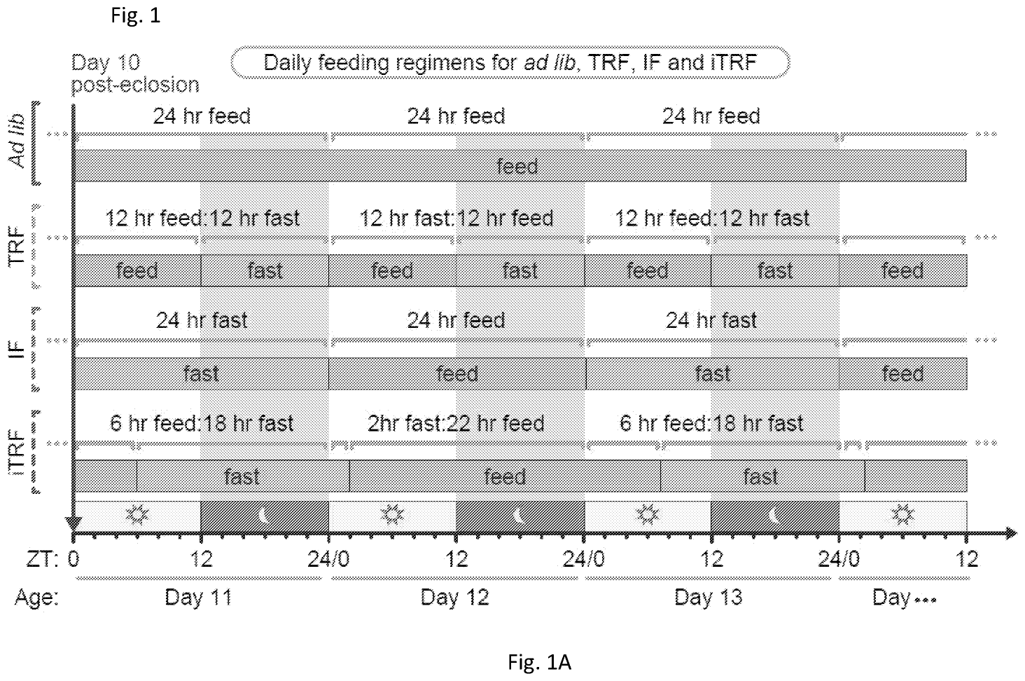

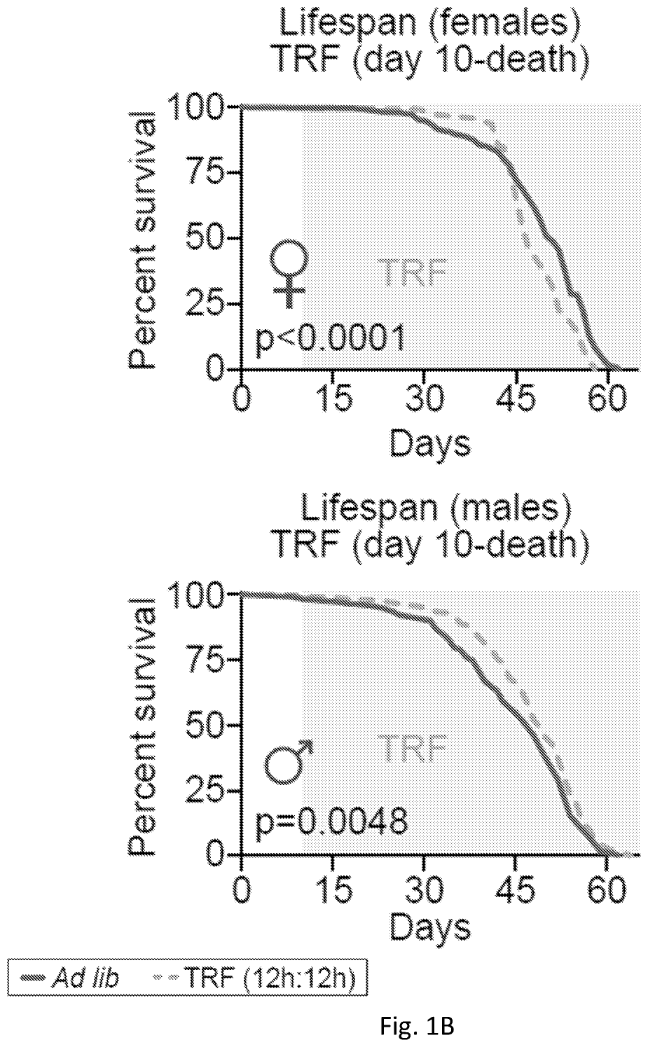

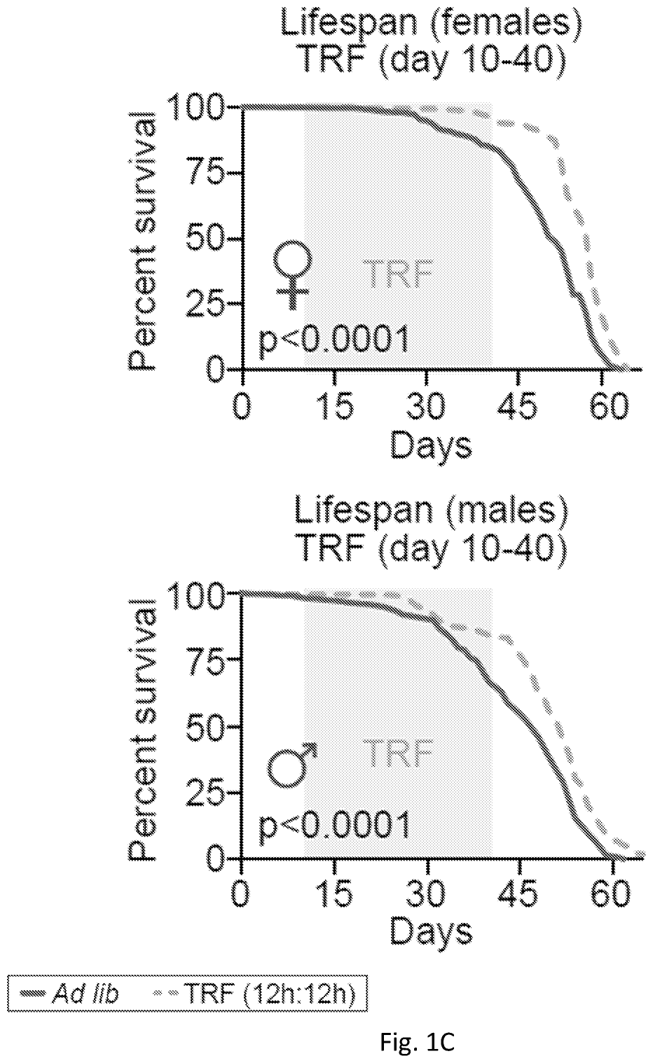

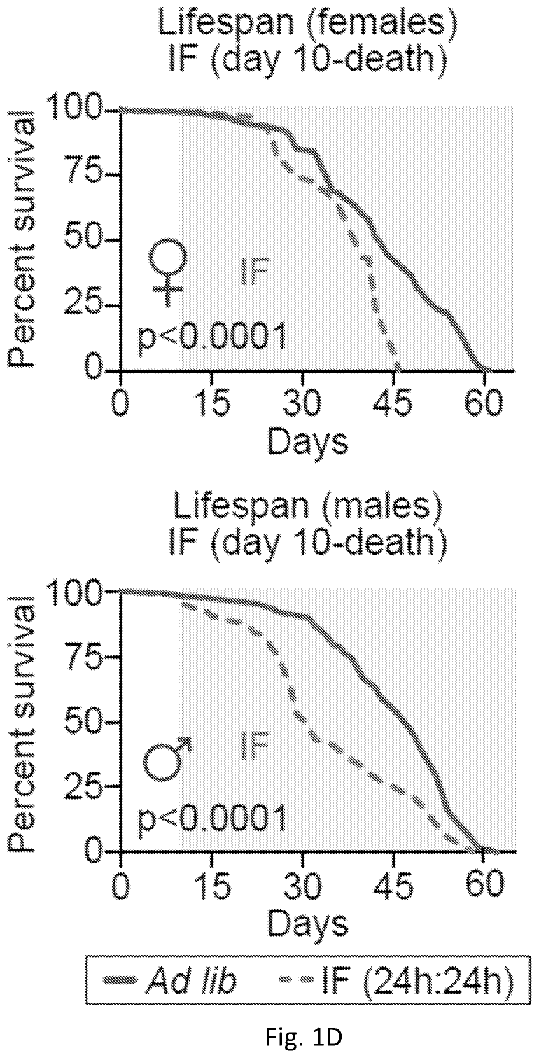

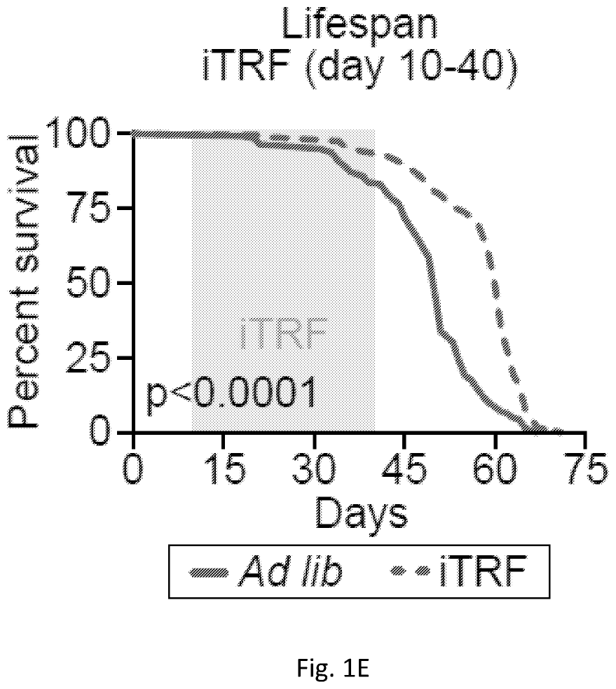

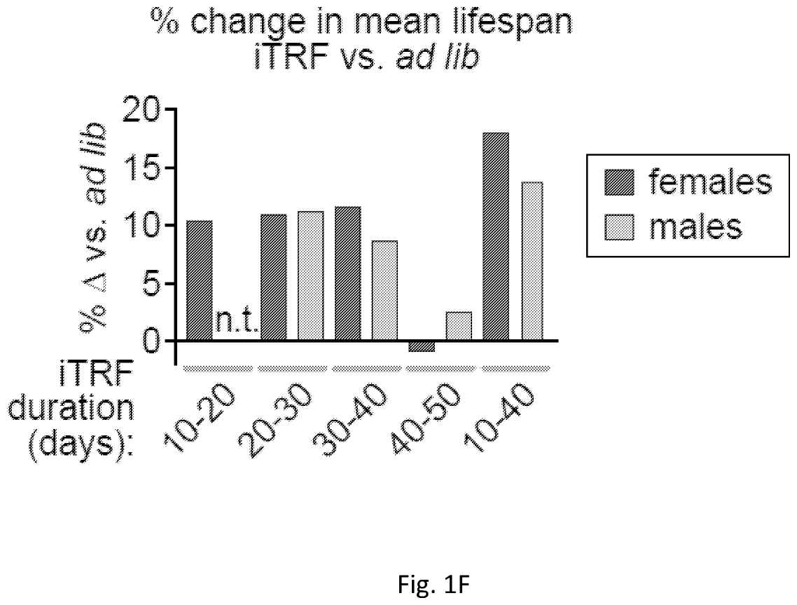

Drosophila were reared from embryos in low-density bottles with standard yeast cornmeal-agar media listed above (Archon Scientific) Newly eclosed flies (˜24 hours) were collected onto adult medium w/3% yeast extract and allowed to mate for 48 hours. Female and male flies were separated and maintained at a density of 30-35 flies per vial in a humidified (65%), temperature-controlled (25° C.) incubator with a 12-hour light-dark cycle. All flies were on ad lib conditions until day 10 upon which flies were placed on various feeding regimes (FIG. 1A)). For consistency, ad lib control flies were flipped on to fresh food at the same time as experimental diet flies were transferred to fasting media, and once again when fasting flies were flipped on to regular adult media. Death was scored at time of flipping, and lifespan compared by log-rank analysis.

### Activity Recording CAFE (ARC) Assay

Feeding data from individual flies were collected as described previously (Murphy et al. 2017; Murphy et al. 2016). The test diet was filtered (0.2-μm cellulose acetate sterile syringe filter, VWR) solution of 4% dextrose, 2% sucrose, and 3% yeast extract in ddH2O. Wild-type (Canton S) female flies were transferred to fresh standard medium every other day until the experiment. When the flies were 9-11 days old, the animals were loaded by mouth pipette into standard ARC chambers and allowed to acclimate overnight (˜18 hours) with access to the test diet in a glass capillary pipette. The capillaries were replaced daily with those containing fresh food or ddH2O. The meniscus level of each capillary was tracked for the entire duration of the feeding periods at 1-second interval. Drops in meniscus position above a precalibrated threshold were considered feeding events and feeding bouts less than 2 minutes apart were considered to be part of the same meal. The volume consumed from each feeding event was automatically calculated and collated with a custom Python code (Murphy et al. 2017; Murphy et al. 2016).

### Intestinal Barrier Dysfunction “Smurf” Assay

The “smurf fly”/intestinal barrier dysfunction assay was performed as previously described (Rera et al. 2012; Rera et al. 2011). Flies were aged on regular medium until the day of the smurf assay. Dyed medium was prepared by the addition of FD&C Blue No. 1 at a final concentration of 2.5% w/v. A fly was counted as a smurf when dye coloration was observed outside the digestive tract. Comparisons of smurf proportion per time point were carried out using binomial tests to calculate the probability of having as many smurfs in population A as in population B, as well as ANOVA for proportions of smurf flies per replicate vial with a minimum of 7 vials of 12-31 flies per replicate.

### Quantitative Real-Time PCR

35-day old female flies on either ad lib or iTRF conditions collected for RNA extraction over the course of 48 hours in 4-hour intervals. 4 biological replicates of 30 female flies per timepoint and feeding regimen were snap-frozen in liquid nitrogen and stored at −80° C. RNA was extracted using TRIzol (Invitrogen) following the manufacturer's protocol. Samples were treated with DNaseI (Invitrogen), then heat inactivated. cDNA was synthesized by Revertaid First Strand cDNA Synthesis Kit (Thermo Scientific). PowerUp SYBR Mastermix (Applied Biosystems) was used to perform qRT-PCR using a CFX-Connect thermal cycler (BioRad). Primer efficiency and relative quantification of transcripts were determined using a standard curve of serial diluted cDNA. Transcripts were normalized using Actin5C as a reference gene. The Jonckheere-Terpstra-Kendall (JTK) algorithm was applied using the JTK-Cycle package in R software (Hughes et al. 2010) to determine significance of rhythmic cycling. Differences between individual timepoints were determined by ANOVA followed by multiple comparison tests.

period-fwd
(SEQ ID NO: 1)
GGTTGCTACGTCCTTCTGGA
period-rev
(SEQ ID NO: 2)
TGTGCCTCCTCCGATATCTT
timeless-fwd
(SEQ ID NO: 3)
CCGTGGACGTGATGTACCGCAC
timeless-rev
(SEQ ID NO: 4)
CGCAATGGGCATGCGTCTCTG
clock-fwd
(SEQ ID NO: 5)
GGATAAGTCCACGGTCCTGA
clock-rev
(SEQ ID NO: 6)
CTCCAGCATGAGGTGAGTGT
atg1-fwd
(SEQ ID NO: 7)
GCTTCTTTGTTCACCGCTTC
atg1-rev
(SEQ ID NO: 8)
GCTTGACCAGCTTCAGTTCC
atg8a-fwd
(SEQ ID NO: 9)
AGTCCCAAAAGCAAACGAAG
atg8a-rev
(SEQ ID NO: 10)
TTGTCCAAATCACCGATGC
actin5C-fwd
(SEQ ID NO: 11)
TTGTCTGGGCAAGAGGATCAG
actin5C-rev
(SEQ ID NO: 12)
ACCACTCGCACTTGCACTTTC
16Sr-fwd
(SEQ ID NO: 13)
AGAGTTTGATCCTGGCTCAG
16Sr-rev
(SEQ ID NO: 14)
CTGCTGCCTYCCGTA

### Microbial Load Quantification

For 16S bacterial rDNA quantification, 4 replicates of 10 whole flies (washed twice in 70% ethanol, and twice in PBS) were used for total DNA extraction via the Power Soil DNA isolation kit (Thermo). Universal primers for the 16S ribosomal RNA gene were against variable regions 1 (V1F) and 2 (V2R), as previously published (Claesson et al. 2010).

### Western Blot Analysis

Whole-body lysates of female flies (30 flies/sample/timepoint) were separated by SDS PAGE using standard procedures. Membranes were probed with antibodies against AMPK phospho-T184 at 1:1000 (Cell Signaling, 40H9); anti-phospho-S6K T398 (Cell Signaling, 9209), and horseradish peroxidase (HRP)-conjugated monoclonal mouse anti-actin antibody at 1:5000 (Sigma-Aldrich, A3854). Rabbit antibodies were detected using HRP-conjugated anti-rabbit IgG antibodies at 1:2000 (Cell Signaling, 7074). Mouse antibodies were detected using HRP-conjugated anti-mouse IgG antibodies 1:2000 dilution (Cell Signaling, 7076). ECL chemiluminescence reagent (Pierce) was used to visualize horseradish peroxidase activity and detected by CCD camera BioRad image station. A minimum of four independent samples of each condition were used for statistical analysis and quantification.

### Immunostaining of Indirect Flight Muscle

Immunofluorescence was performed as previously described (Rana et al. 2013). Hemithoraces were dissected and fixed for 20 minutes in PBS with 4% paraformaldehyde and Triton X-100. After washing, samples were incubated overnight at 4° C. with an antibody against poly-ubiquitinated proteins at 1:200 mouse mAb FK2 (Enzo) and 1:200 anti-P62 rabbit ab178440 (Abcam) then washed thoroughly and incubated with secondary anti-mouse Alexa-488 (1:250) Alexa-555 (1:250) and phalloidin Alexa-647 (1:150). Samples were rinsed three times in PBS+0.1% triton X-100 for 10 minutes at room temperature, then mounted in Prolong Gold (Invitrogen) and imaged by confocal microscopy (Zeiss). For quantification of protein aggregates of hemithoraces, the size and area of protein aggregates was measured using ImageJ particle counter software (Schneider et al. 2012). Statistical analysis was conducted using a two-tailed, unpaired Student's t-test or ANOVA followed by Tukey's multiple comparisons (n≥9 thoraces per condition).

### Climbing Activity

Assessment of climbing ability was performed as previously described (Copeland et al. 2009) with minor modifications. Briefly, 10 flies were transferred to an empty standard 23 mm×95 mm plastic vial and then gently tapped to the bottom. The number of flies that reached the top ¼ of the vial within 20 seconds were then scored as climbing. Each experiment was performed on a minimum of 8 vials of 10 flies per condition repeated three times.

### Lysotracker Red Staining

35-old female flies that were nearly done with a 20-hour fast ˜18 hours of iTRF were anesthetized on ice and intestines were dissected in cold PBS. Intestines were washed once in PBS, followed by three 30-second rinses in freshly prepared 1 μM Lysotracker Red, (Invitrogen) and 1.5 μg/mL Hoechst stain in PBS at room temperature. Intestines were washed five times for 30 s in PBS at room temperature, then mounted in Vectashield, and imaged immediately. Imaging should not proceed for very long as apoptosis can be observed after approximately 60 minutes. Colocalization GFP-atg8a quantification was conducted in COLOC2 plugin for ImageJ (Schneider et al. 2012). The number of vesicles with significant Lysotracker and GFP-Atg8a colocalization determined by significant Pearson's Correlation Coefficient were then counted using the particle counter tool. Similarly, the total number of lysotracker and GFP-Atg8a vesicles were determined using the particle counter tool. Statistical analysis was conducted using a two-tailed, unpaired Student's t-test.

### Triton-Insoluble Protein Extracts

Flies were homogenized in ice-cold PBS with 1% Triton X-100 and protease inhibitor cocktail (Roche). The mixture was spun for 10 minutes at 4° C., and the pellet and supernatant were collected. The Triton X-100—insoluble pellet was washed in one additional volume of Triton X-100 solution and resuspended in denaturing lysis solution with 5% lithium dodecyl sulfate (LDS) containing 300 mM dithiothreitol (NuPAGE LDS Sample Buffer; Invitrogen). N=4 independent samples of 30 flies were used for postwestern blot densitometry analysis.

### Phospho-Histone H3 Immunostaining

Briefly, flies were anesthetized on ice and intestines were dissected in cold PBS. Samples were then fixed in PBS+0.1% Triton X-100 containing 4% paraformaldehyde at room temperature for 30 minutes and rinsed three times in PBS+0.1% Triton X-100 for 10 minutes at room temperature. Blocking was performed in 5% BSA in PBS+0.1% triton X-100 for one hour at room temperature. Primary antibody, anti-phospho-histone H3 (S10) (Cell Signaling, 9701), was added 1:250 in 5% BSA in PBS+0.1% triton X-100 and incubated overnight at 4° C. rotating. After washing three times in PBS+0.1% Triton X-100 secondary antibody, anti-rabbit AlexaFluor-488 (Invitrogen) was added 1:250, and 1.5 μg/mL Hoechst stain (Thermo) in 5% BSA in PBS+0.2% triton X-100 and incubated overnight at 4° C. rotating. After washing, intestines were then mounted in Vectashield mounting medium (Vector Labs) and Imaged using Zeiss LSM800. Phospho-histone H3 (p-HH3) positive cells were quantified using the ImageJ local maxima tool, with identical thresholding for all images. p-HH3 numbers were normalized to the area of the posterior midgut imaged. A minimum of 8 intestines were used for each quantification.

### Statistical Analysis

Prism? (GraphPad) was used to perform the statistical analysis. Significance is expressed as p values (ns=p>0.05, *=p<0.05, **=p<0.01, ***=p<0.001). For two group comparisons unpaired, two-tailed t-test was used, when data met criteria for parametric analysis (normal distribution and similar variance). For more than two group's comparison ANOVA with Bonferroni, or Tukey' s post-hoc test was performed.

Kruskal-Wallis with Dunn's post-hoc for data that was not distributed appropriately for the parametric comparisons. For comparison of survival curves, Log-rank (Mantel-Cox) test was used.

### Example 2—Intermittent Time-Restricted Feeding (iTRF) Extends Drosophila Lifespan

A time-restricted feeding schedule for Drosophila was developed that provides both health benefits and lifespan extension. To do this, four different feeding schedules were tested, biased towards daytime feeding (FIG. 1A). For the control diet, flies were allowed 24-hour access to food and ate “at will” (ad libitum or ad lib). The standard time-restricted feeding (TRF) schedule (12 hours of ad lib diet during the day and 12 hours of fasting during the night) did not extend lifespan relative to control diet unless it was limited to days 10-40 of lifespan; even then, as seen previously, TRF-mediated lifespan extension was modest (FIGS. 1B and 1C) (Villanueva et al. 2019; Catterson et al. 2018). In contrast, longer fast periods used in standard intermittent fasting schedules (24-hour fast beginning at lights on or ZT0, followed by 1-2 days of ad lib feeding and recovery) did not extend lifespan and in fact, as seen previously, shortened lifespan (FIG. 1D) (Catterson et al. 2018; Li et al. 2017).

An intermediate feeding schedule was discovered that robustly extended lifespan (FIG. 1A). Flies fasted for 20 hours every other day, starting at mid-morning (6 hours after lights on or ZT6), with a recovery day of normal feeding (ad lib diet) between fast days. While maintaining this diet through old age did not extend lifespan, maintaining this diet for a limited 30-day window from 10-40 days old did lead to consistent, significant lifespan extension (FIG. 1D). Application of this diet for shorter 10-day windows at different ages (days 10-20, 20-30, or 30-40) extended lifespan incrementally; application of this diet for ten days in old flies (days 40-50 of adulthood) gave no lifespan benefit (results not shown). Relative to animals on ad lib diets, animals on this diet from days 10-40 had mean lifespan increase of >18% (females) and 13% (males) (FIG. 1F).

Therefore, the following experiments focused on females. This lifespan-extending dietary regimen was designated “intermittent time-restricted feeding”, or iTRF.

### Example —iTRF-Mediated Lifespan Extension Was Not Due to Nutrient Restriction or Modulation of Insulin Signaling

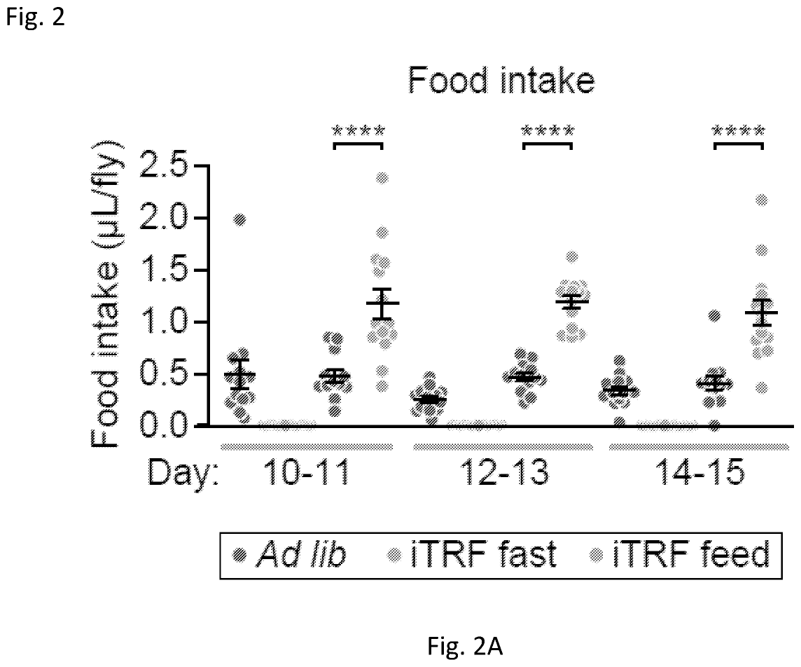

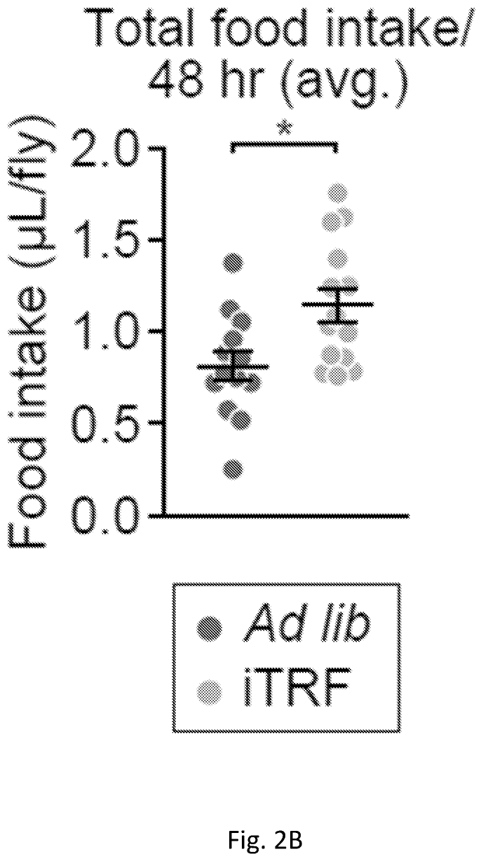

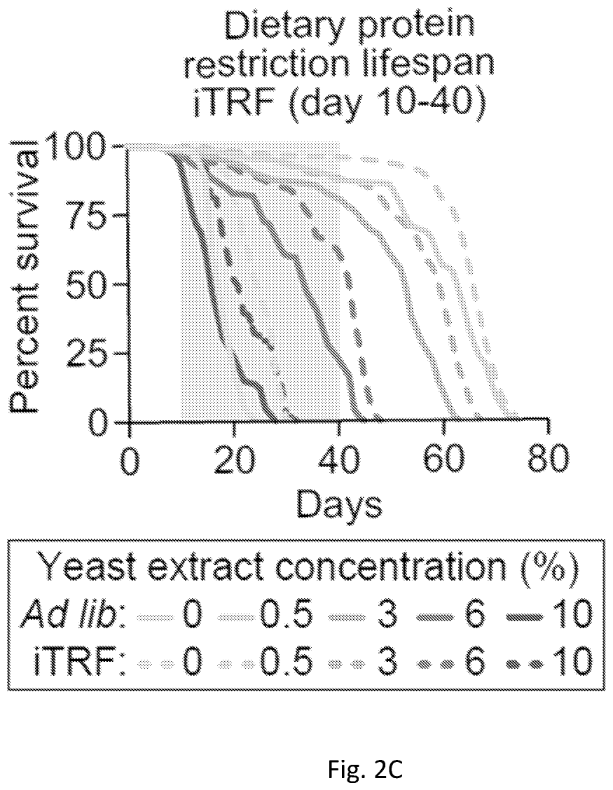

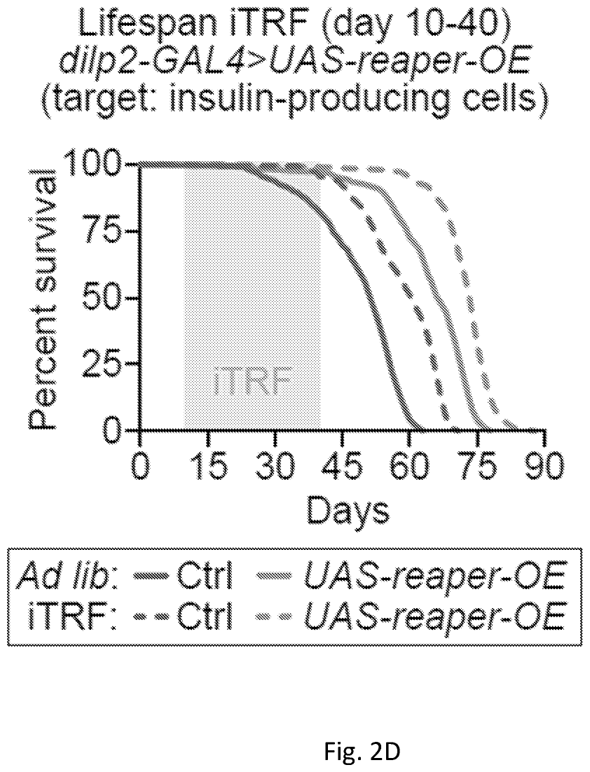

Caloric restriction, or decreased food intake, is known to extend lifespan, and it was possible that the health benefits of the iTRF regimen were due to caloric restriction (Longo and Panda 2016). To test food intake, the feeding rates of flies on ad lib and iTRF diets were measured over 3 cycles of fasting and refeeding via the CAFE assay (Ja et al. 2007; Murphy et al 2017). iTRF flies exhibited compensatory feeding during the recovery period (FIG. 2A), resulting in slightly increased average food consumption over 48 hours (fast day plus feed day), relative to the same 48-hour period for control animals on ad lib schedule (FIG. 2B). Thus, iTRF does not extend lifespan by limiting nutrient intake.

In flies, two well-established lifespan-extending manipulations are dietary protein restriction (DR or reducing protein intake), and inhibition of insulin-like signaling (Partridge et al. 2011). To test whether iTRF acts via DR, flies were put on different protein concentration diets (including DR) and combined those with either ad lib or iTRF feeding schedules (FIG. 2C). If iTRF acts solely via DR, it would be expected that iTRF would not extend lifespan further than DR alone. Relative to the ad lib control feeding schedules, it was found that iTRF significantly extended lifespan with every protein concentration tested, including DR diet (0.5% yeast extract). Thus, iTRF can function independently of DR-mediated longevity extension.

Similarly, inhibition of insulin-like signaling, a conserved longevity pathway, allowed for typical iTRF-mediated lifespan extension of both controls and iTRF flies to similar magnitudes (FIG. 2D).

Together these results suggested that iTRF flies were not long-lived due restricted nutrient intake or inhibition of the insulin signaling pathway.

### Example 4—iTRF Extends Healthspan and Delays Aging

To assess healthspan and anti-aging effects of iTRF, known age-related changes were assayed in muscle/neuronal function, protein aggregation, and intestinal function.

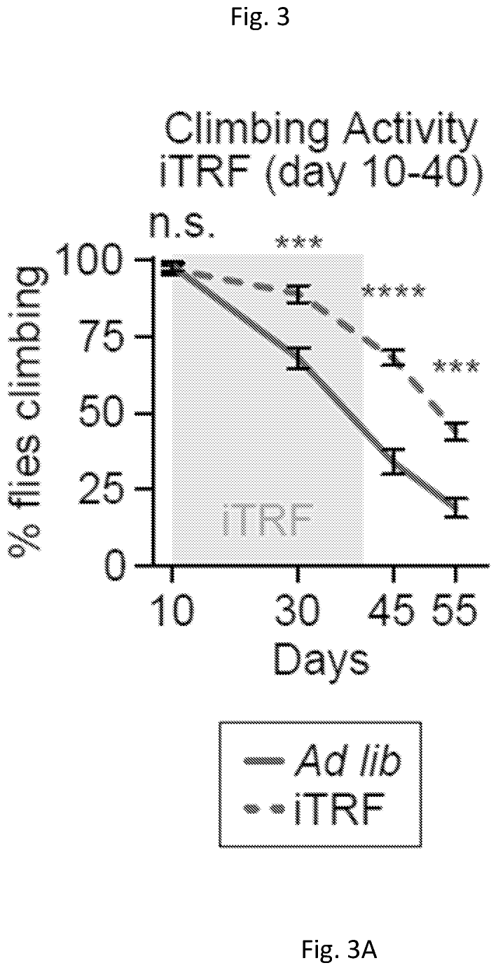

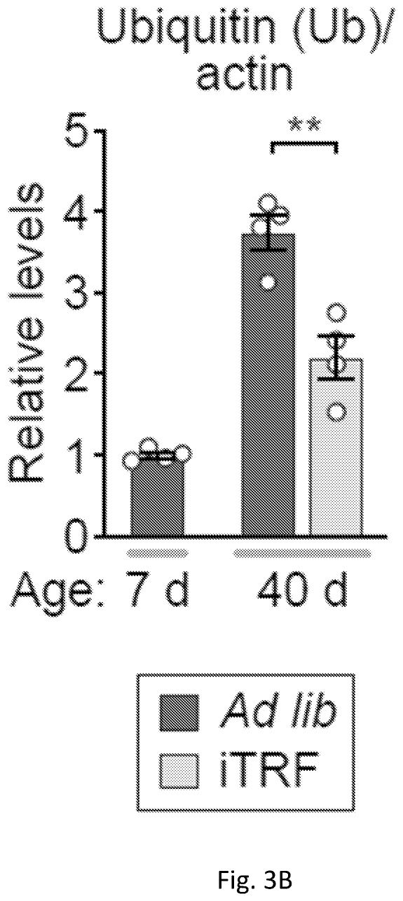

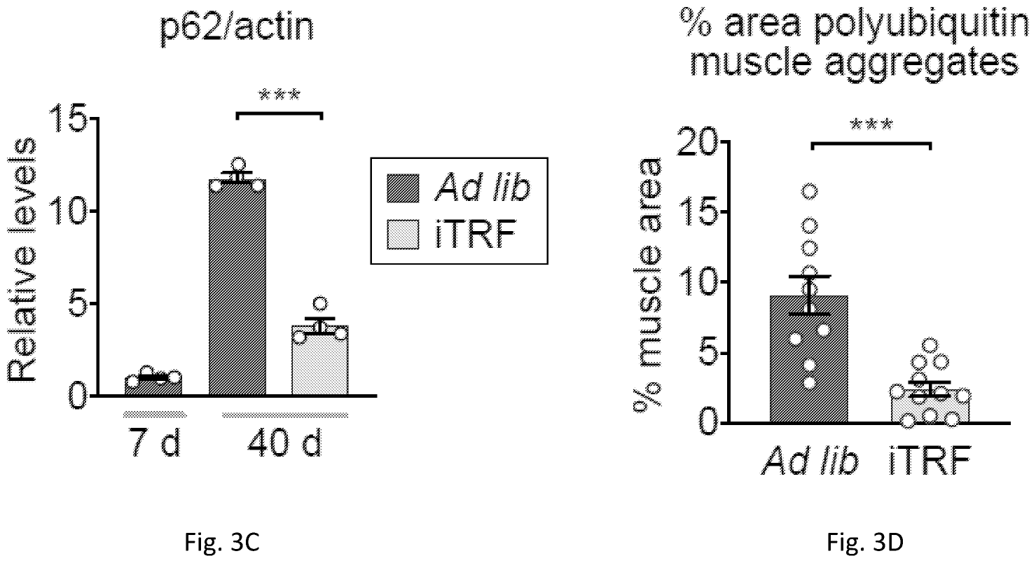

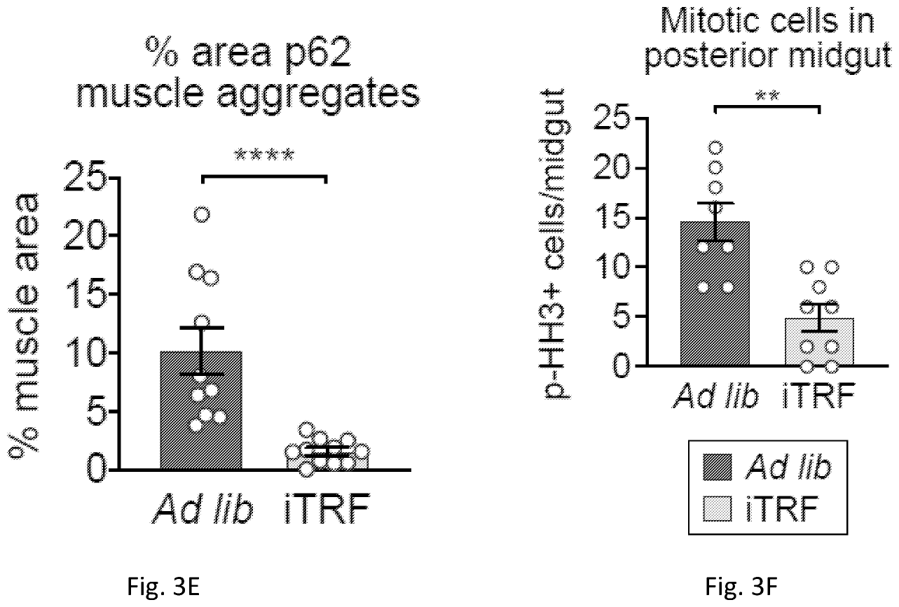

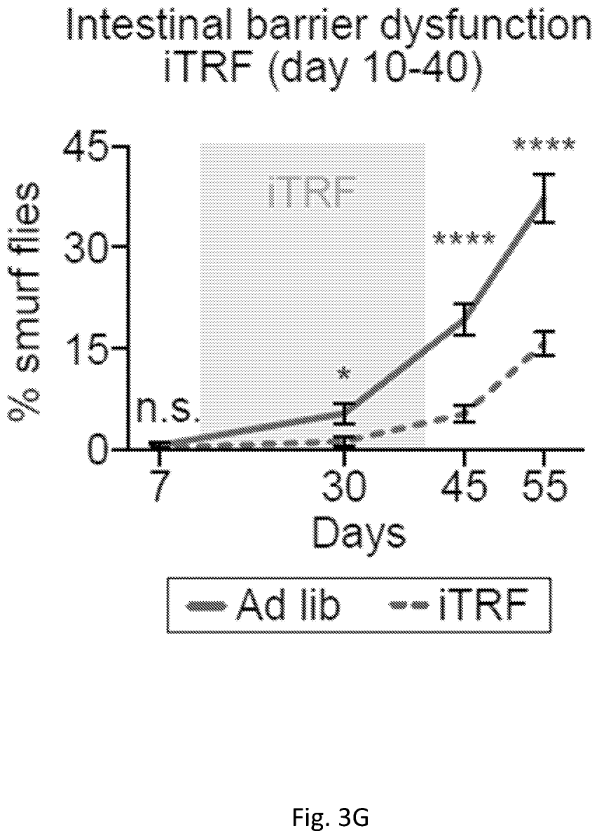

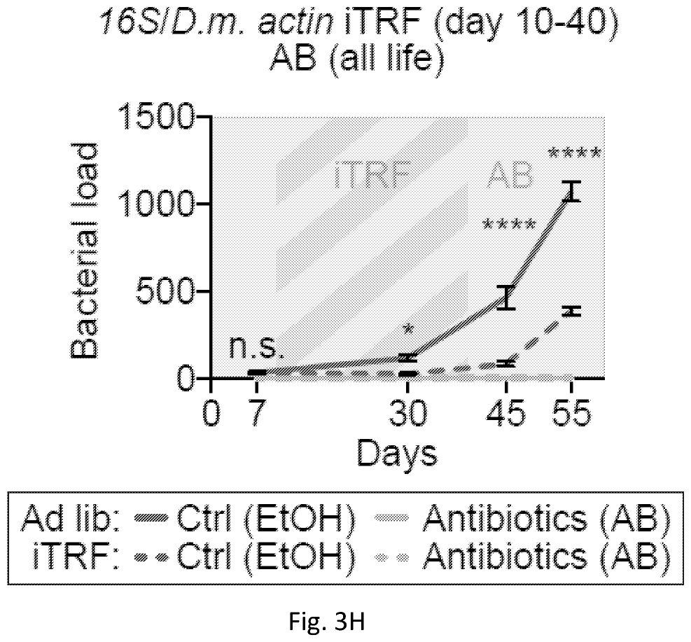

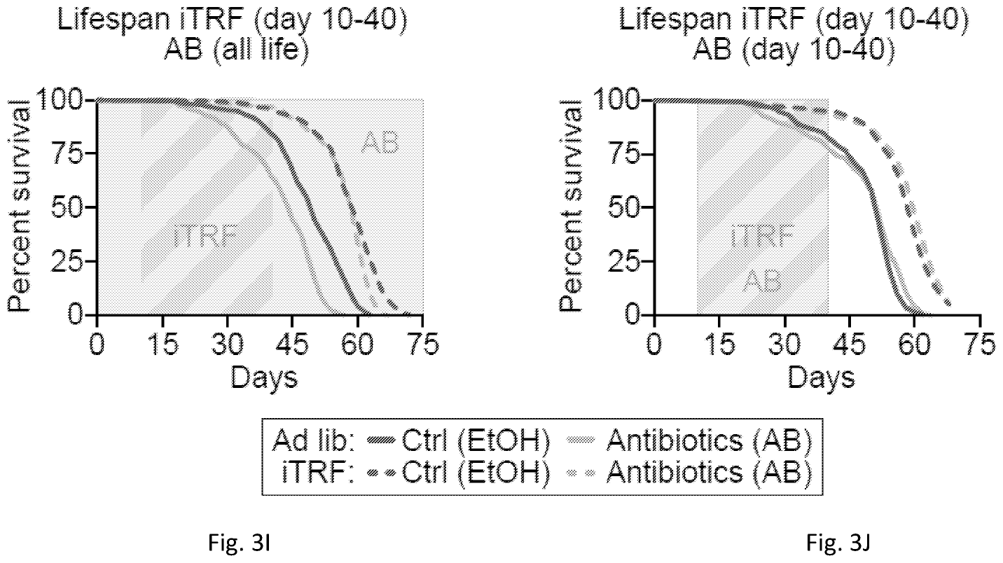

First, to assess overall muscle/neuronal function, climbing ability was assayed. iTRF flies exhibited a slower decline in climbing ability with age relative to ad lib flies (FIG. 3A).

Second, to measure conserved markers of age-related protein aggregation, Triton-insoluble fractions from whole-fly extracts of iTRF and ad lib flies were extracted both pre- and post-iTRF intervention (7 and 40 days old) followed by Western blot analysis for ubiquitin (FIG. 3B) and Drosophila ortholog of p62/SQSTM1, hereafter referred to as just p62 (FIG. 3C). For both markers, flies on iTRF had decreased levels in the insoluble fraction relative to ad lib control diet, demonstrating less aging-related protein aggregation.

Third, protein aggregation in muscle was directly measured using anti-polyubiquitin and anti-p62 antibodies with confocal immunofluorescence microscopy. Again, relative to ad lib control diet, iTRF significantly decreased the number and area of polyubiquitin and p62 aggregates in the flight muscle of aged flies (FIGS. 3D and 3E).

Fourth, because decreased intestinal function is another conserved marker of aging, aging-related intestinal stem cell over-proliferation, intestinal integrity and intestinal microbial load were measured. iTRF decreased these intestinal markers of aging relative to ad lib control diet (FIGS. 3F-3H).

Finally, because dietary changes impact intestinal microbes, which can concomitantly impact aging, it was determined if iTRF-mediated lifespan extension depends on fly-associated microbes. Treatment with a cocktail of antibiotics during both fasting and feeding phases depleted intestinal microbes to nearly non-detectable levels (FIG. 3H). iTRF caused the same lifespan extension of antibiotic-treated flies as ad lib controls, suggesting that iTRF does not depend on fly-associated microbes (FIGS. 3I and 3J).

Together, these results demonstrated that iTRF decreases multiple conserved parameters of aging relative to ad lib control diet and suggested that iTRF extends lifespan by increasing healthspan and delaying aging.

### Example 5—Core Circadian Clock Components Were Required For iTRF-Mediated Lifespan and Healthspan Extension

Because iTRF does not alter dietary composition or reduce food intake and instead controls the timing of feeding, the role of circadian clock components was examined, which regulate the timing of physiological functions such as metabolism.

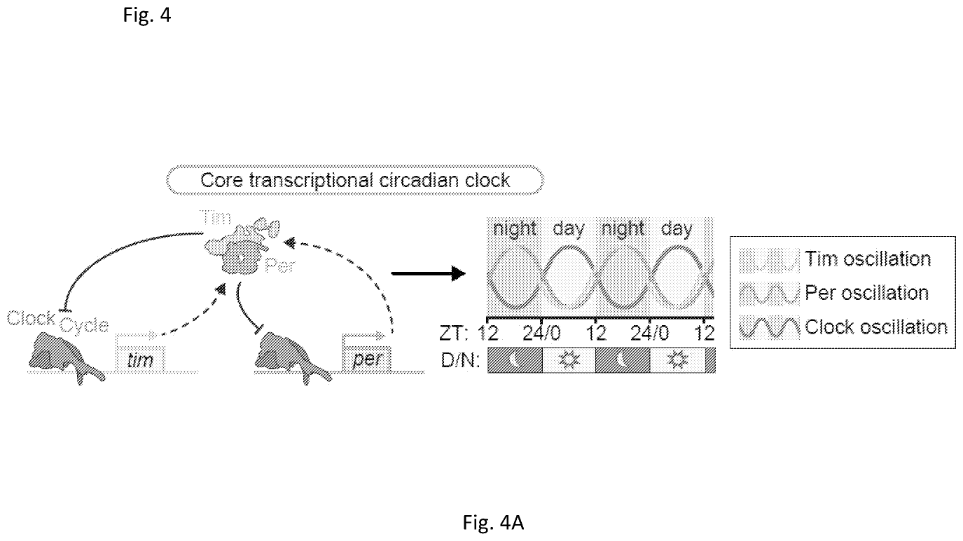

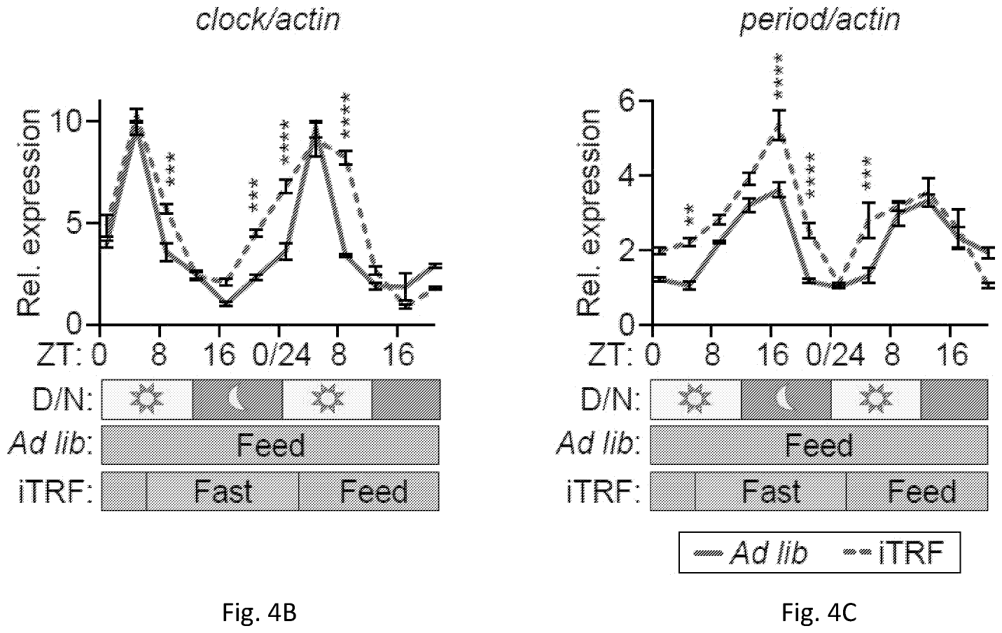

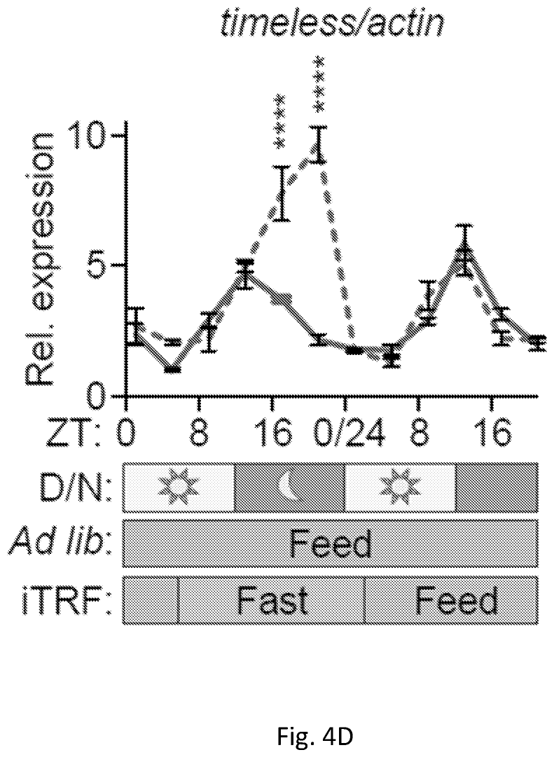

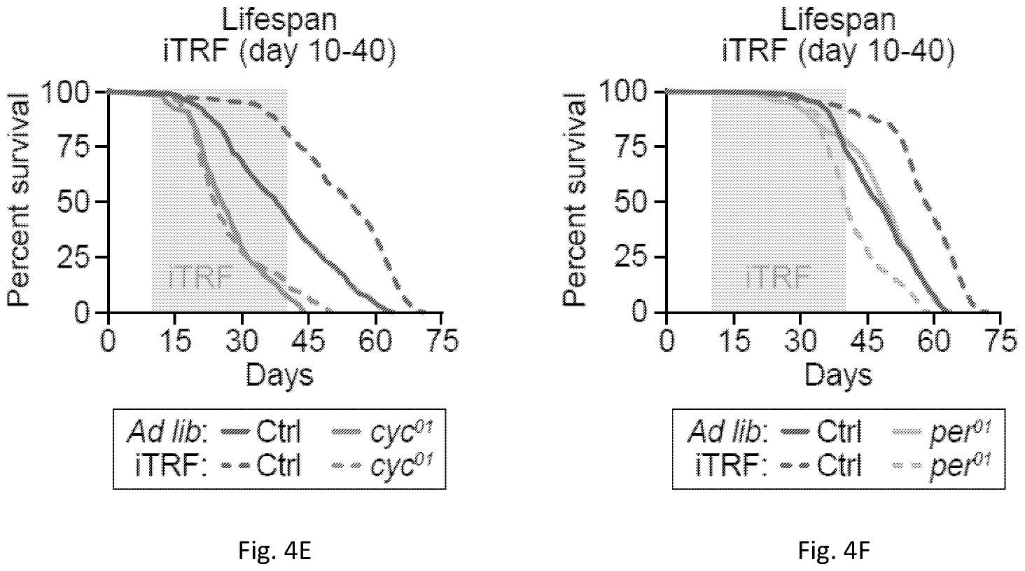

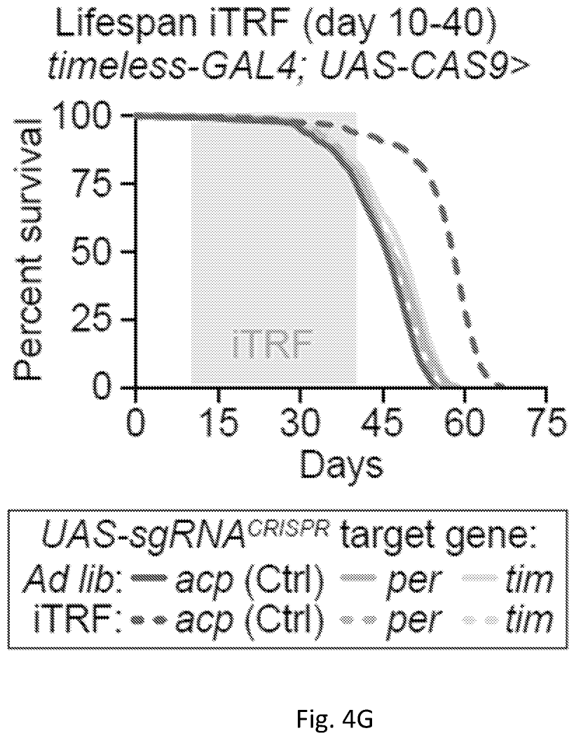

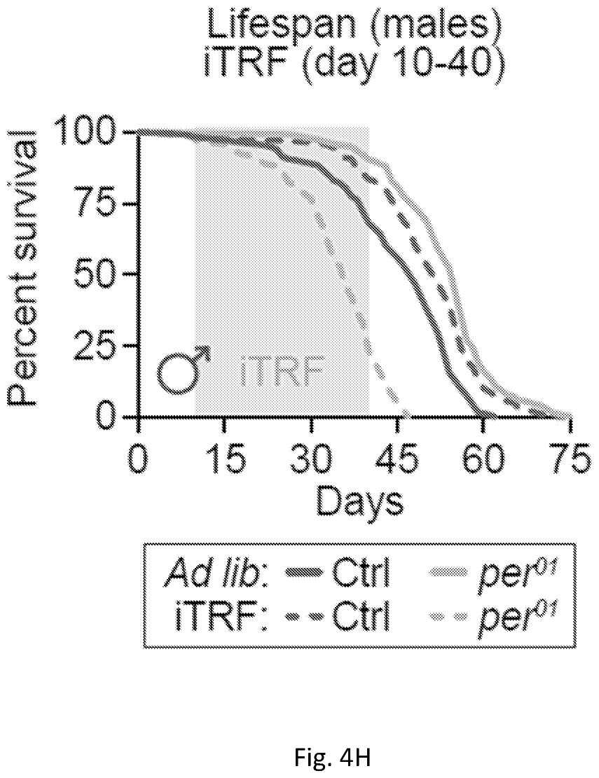

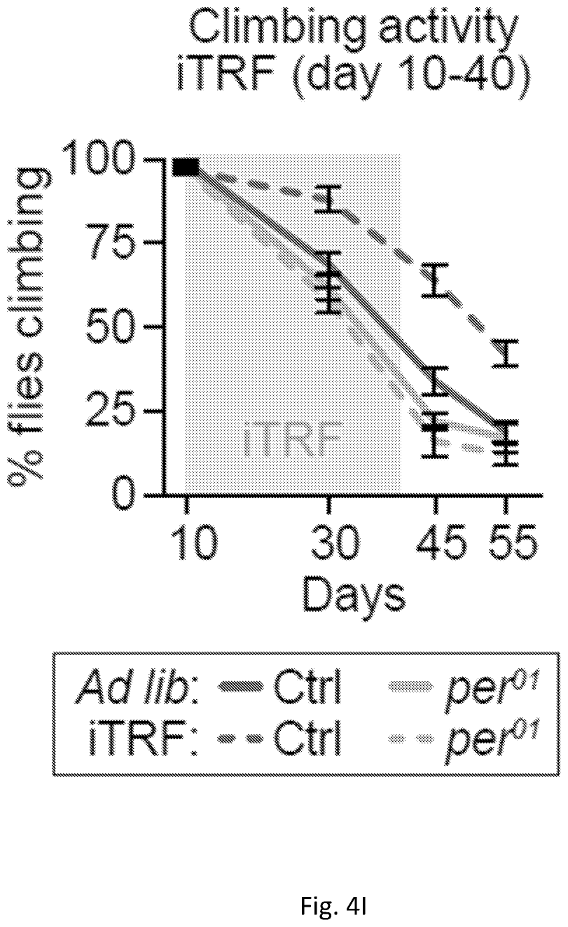

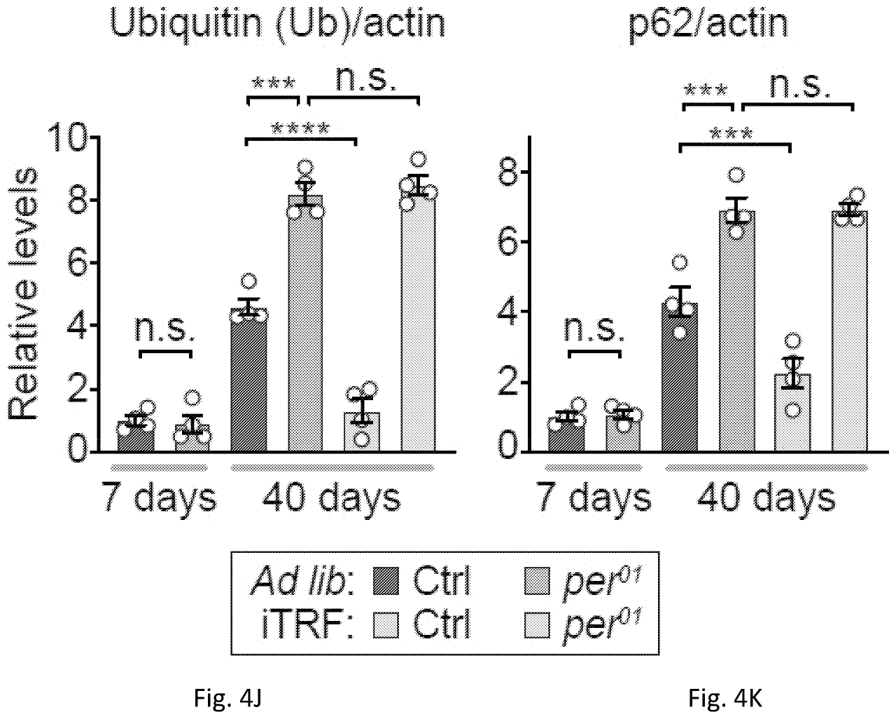

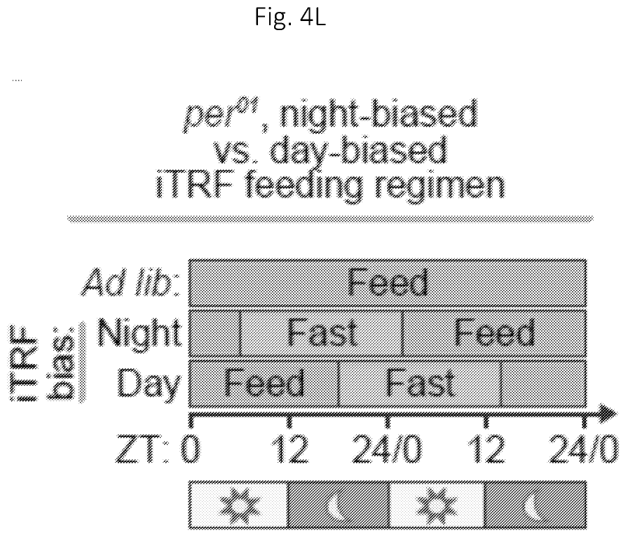

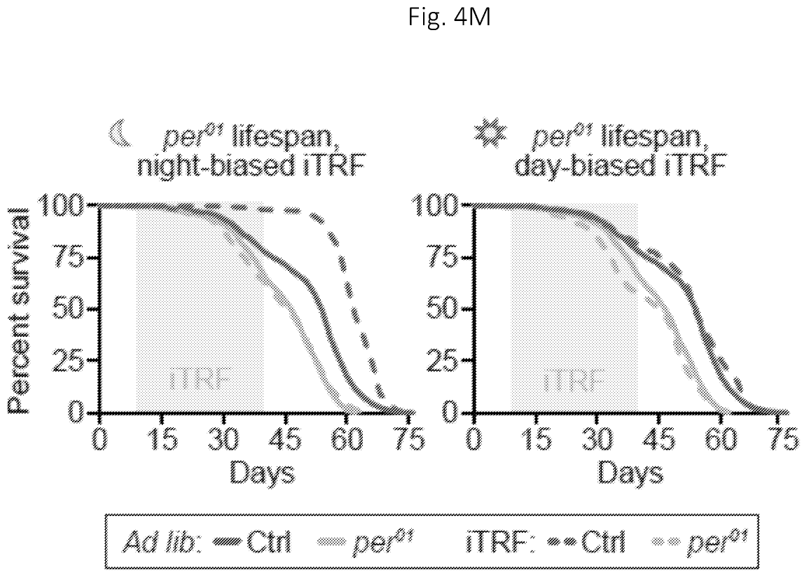

Circadian clock components are highly conserved across almost all animals and regulate 24-hour oscillations in transcription, which underlie 24-hour oscillations in physiology and behavior (Allada and Chung 2010; Panda 2016). The core circadian clock components form a transcriptional negative feedback loop: in Drosophila, Clock (Clk) and Cycle (Cyc) proteins activate the transcription of hundreds of genes, including the circadian regulators, period (per) and timeless (tim), whose gene products inhibit Clock and Cycle activity (FIG. 4A), and multiple metabolic genes that may underlie TRF health benefits.

Others have hypothesized that TRF enhances circadian oscillations in gene expression, based on the observation that standard 12:12 TRF increases circadian gene expression (Longo and Panda 2016). To determine the effect of iTRF on core circadian clock gene expression, qRT-PCR analysis was performed on RNA isolated from animals on ad lib control and iTRF diets every four hours for 48 hours. iTRF increased the amplitude of period and timeless gene expression, specifically during the fasting phase (FIGS. 4C and 4D) and broadened the peak of clock expression (FIG. 4B). Thus, the iTRF regimen enhanced circadian gene expression.

It is not known if circadian clock components are required for iTRF-mediated lifespan extension. To test this, the effects of ad lib and iTRF diets on several arrhythmic circadian mutants and their genetic controls, including cycle mutants (cyc⁰¹), period mutants (per⁰¹), and mutants containing CRISPR-mediated disruption of period (per^{CRISPR}) or timeless (tim^{CRISPR}) (Delventhal et al. 2019; Ulgerait et al. 2016). While genetic controls exhibited significant lifespan extension on iTRF, circadian mutants did not (FIGS. 4E-4G), including cyc⁰¹ mutants, which have shorter lifespans than genetic controls, and male per⁰¹ mutants, which have longer lifespans than genetic controls (FIG. 4H) (Ulgerait et al. 2020). It was further confirmed that, similar to control animals, per⁰¹ and cyc⁰¹ mutants have a normal response to DR (results not shown) (Ulgerhait et al. 2016; Ulgerait et al. 2020). Because circadian mutants specifically do not respond to iTRF, these results suggested that iTRF-mediated lifespan extension requires a functional circadian clock.

To test if iTRF-mediated healthspan extension is circadian clock-dependent, two aging parameters (climbing ability and accumulation of ubiquitinated proteins) were measured for young and old per⁰¹ mutants and controls on iTRF or ad lib diets. While iTRF preserved climbing ability and lowered ubiquitinated protein levels in genetic controls relative to ad lib diets, per⁰¹ mutants did not respond to iTRF and exhibited aging-related declines in climbing ability and increased protein aggregation similar to per⁰¹ mutants on an ad lib diet (FIGS. 4I-4K). Thus, consistent with iTRF-mediated lifespan extension depending on healthspan extension, both iTRF-mediated lifespan and healthspan extension required a functional circadian clock.

To test if iTRF-mediated lifespan extension requires a night-biased fast period, a day-biased iTRF, shifted by 12 hours (ZT18 to ZT14 the following day) was tested, with mainly daytime-fasting (FIG. 4L). While night-biased iTRF significantly extended lifespan relative to ad lib controls, day-biased iTRF did not have as great an effect (FIG. 4M). per01 mutants were unaffected by either iTRF regimen. This result suggests that night-biased fasting may be better for iTRF lifespan extension.

### Example 6—Circadian-Regulated Key Autophagy Components Are Required For iTRF-Mediated Lifespan Extension and Healthspan Extension

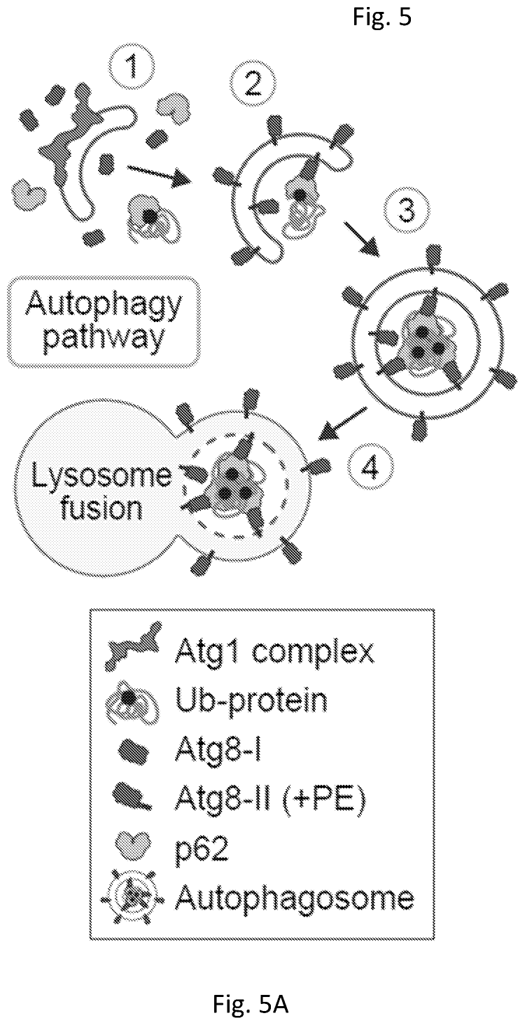

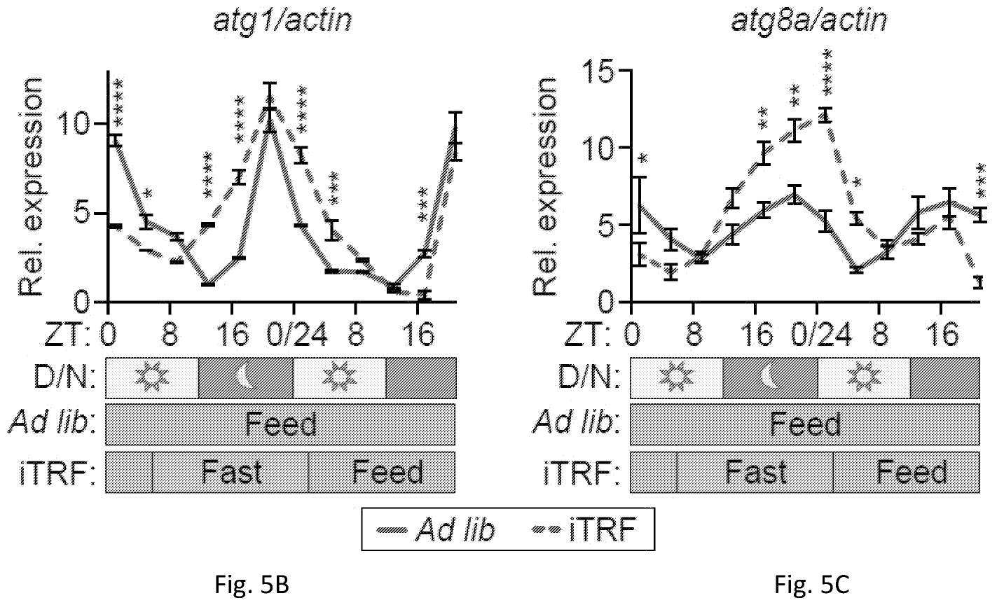

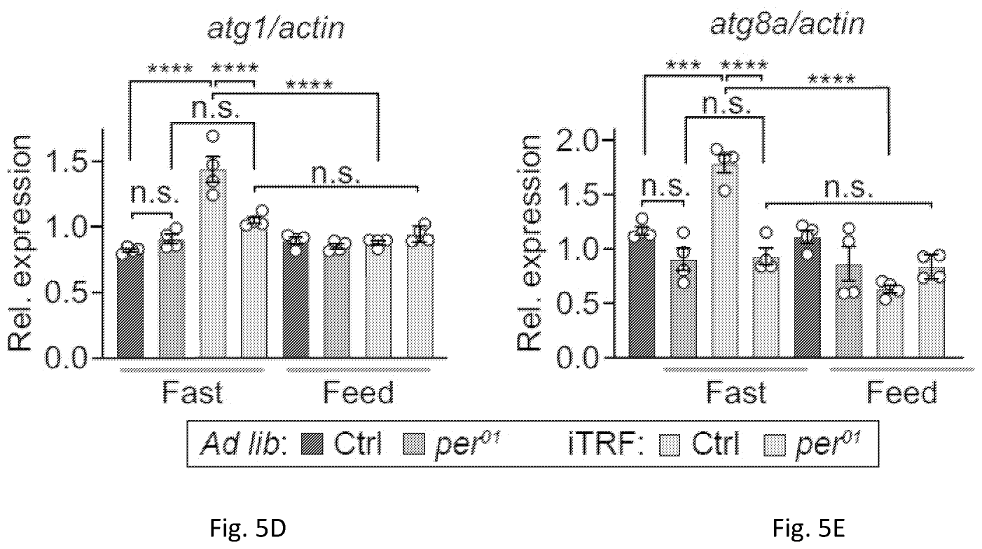

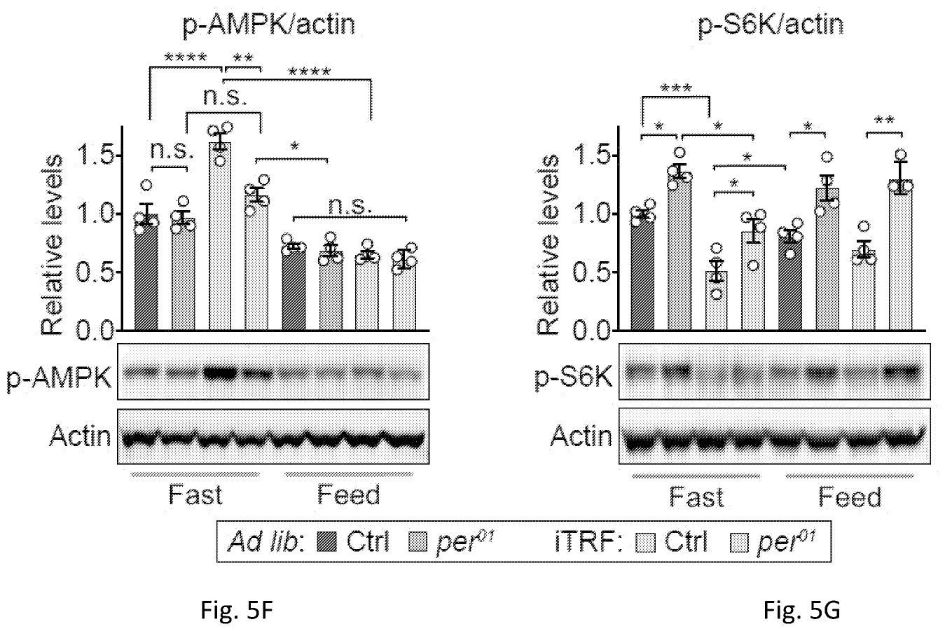

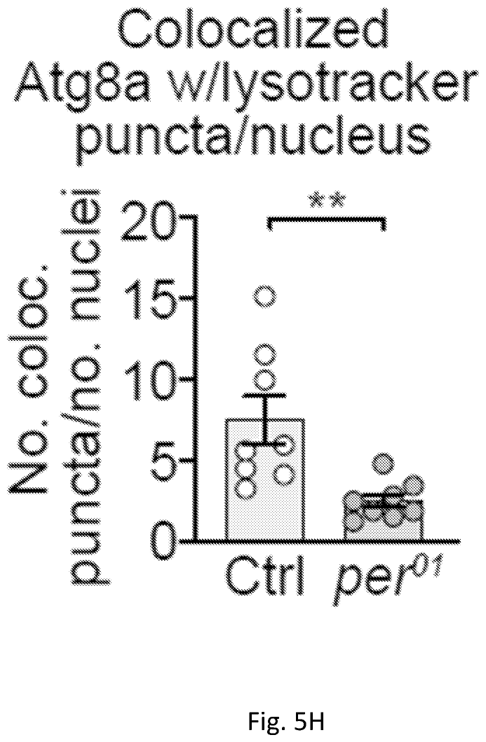

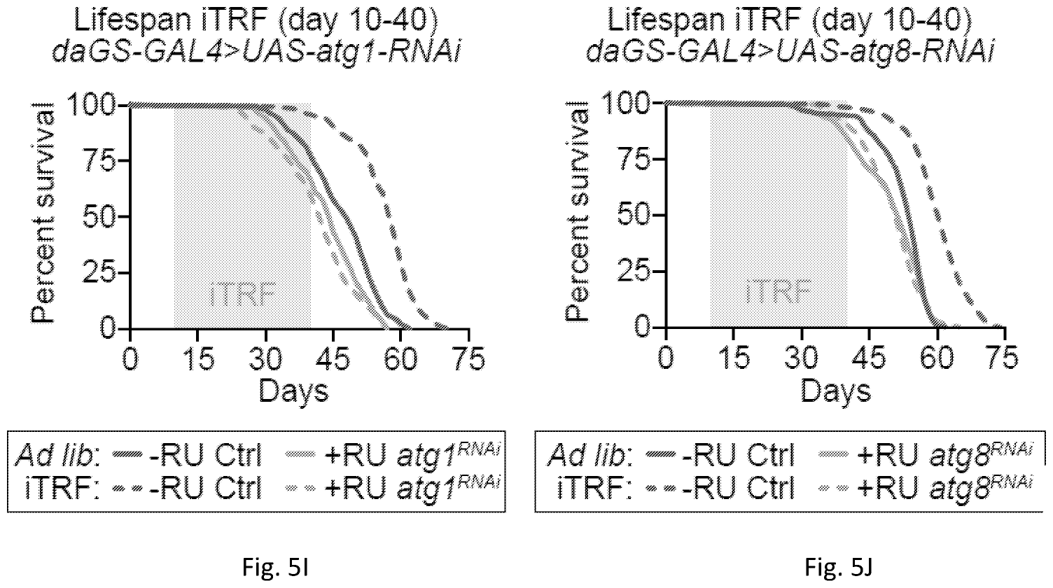

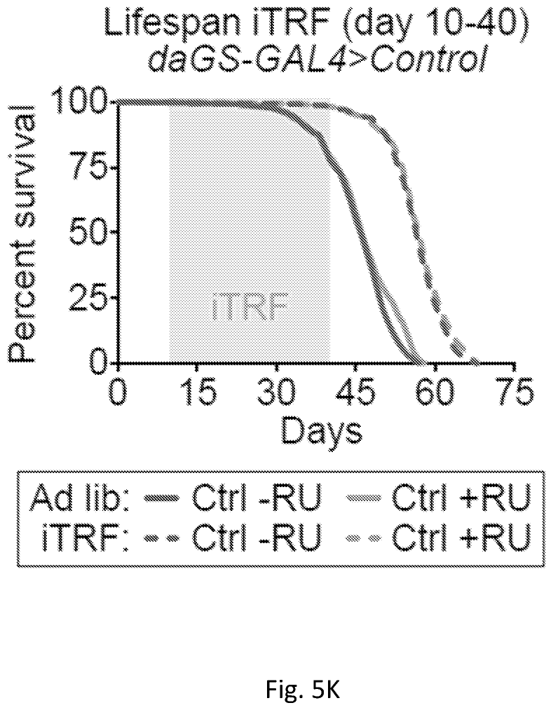

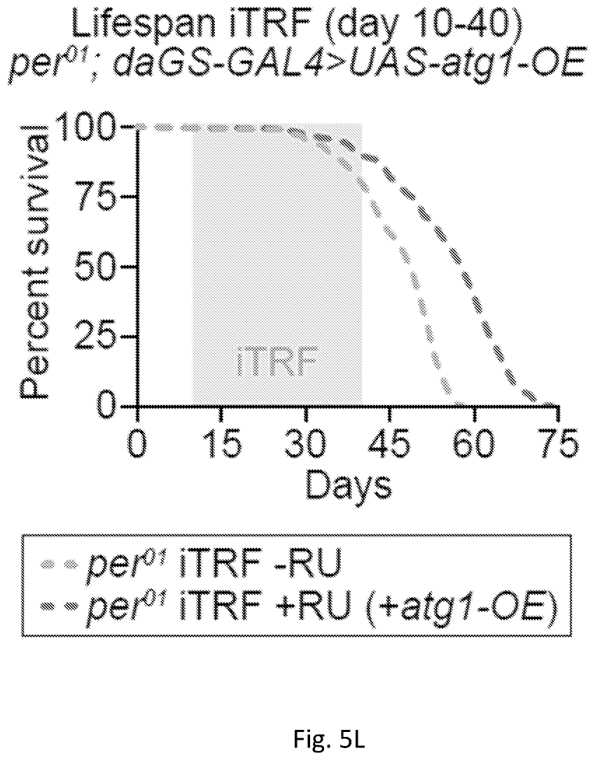

Because time-restricted feeding involves a fasting period, the role for autophagy, a starvation-induced cellular process to recycle macromolecules, was examined (FIG. 5A).

First, it was determined if conserved components of autophagy (Atg1 and Atg8, whose mammalian homologs are ULK1 and LC3, respectively) are circadian-regulated in Drosophila. Consistent with other model organisms and cell culture results (Kalfalah et al. 2016; Ma et al. 2012), both atg1 and atg8 were circadian-regulated in wild-type flies (FIGS. 5B and 5C solid lines). The circadian peaks of atg1/atg8 gene expression coincided with those of period and timeless.

It was next determined if iTRF affects atg1 and atg8 gene expression. Indeed, it was found that iTRF increased the peak amplitude of atg1 and atg8 expression, specifically during the fasting period (FIGS. 5B and 5C, dashed lines). In contrast, iTRF did not increase atg1 or atg8 expression in per⁰¹ mutants, consistent with their lack of iTRF-mediated lifespan extension (FIGS. 5D and 5E). Thus, the expression levels of these two key autophagy genes were both circadian-regulated and enhanced by iTRF.

To determine if iTRF induces autophagic processes, autophagy protein levels were measured, signaling markers of autophagy, and autophagosome/autolysosome information. iTRF was compared to ad lib diet for both genetic control animals and per⁰¹ mutants, predicting that, if autophagy is the circadian-regulated function underlying iTRF-mediated lifespan extension, per⁰¹ mutants would exhibit lessened iTRF-induced autophagic function.

Two molecular markers of activated AMPK and reduced TOR signaling associated with induction of autophagy were examined: increased phosphorylation of AMPK and decreased phosphorylation of downstream target S6-Kinase (S6K) (Rubinstein et al. 2011). Consistent with the hypothesis that iTRF enhances circadian-regulated autophagy, iTRF increased levels of phospho-AMPK and decreased levels of phospho-56K relative to ad lib diets for controls but not per⁰¹ mutants, specifically during the fasting period (FIGS. 5F and 5G).

Also, autophagosome formation, lysosome abundance, and autolysosome formation was directly monitored in an easily dissected tissue, the intestine, via live fluorescent imaging of the autophagosome marker GFP-Atg8 relative to the lysosomal marker lysotracker. Again, consistent with iTRF-mediated induction of autophagy, iTRF increased levels of active autolysosomes in controls but not per⁰¹ mutants (FIG. 5H). Thus, iTRF-mediated induction of autophagy was correlated with lifespan extension and depends on a functional circadian clock.

To determine if these autophagy components are required for iTRF-mediated lifespan extension, atg1 and atg8a expression was manipulated using UAS-RNAi mediated knockdown via a ubiquitous inducible driver (daughterless-GeneSwitch-GAL4 or daGS-202 Gal4) (Tricoire et al. 2009). Flies were fed either the inducing drug RU486 or vehicle control from day 5 post-eclosion until death. Knockdown of either atg1 or atg8a expression prevented the iTRF-mediated lifespan extension (FIGS. 5I and 5J). RU486 feeding had no effect on iTRF2 mediated lifespan extension of flies lacking UAS-RNAi transgenes (FIG. 5K). These data suggested that atg1 and atg8a are required for iTRF-mediated longevity.

Because per⁰¹ mutants lack iTRF-induced autophagy gene expression and function (FIGS. 5D-5H), it was hypothesized that expressing a gene that induces autophagy could allow per⁰¹ mutants to respond to iTRF. To test this, Atg1 was over expressed ubiquitously at low levels in per⁰¹ mutants and the lifespan response to iTRF was measured (FIG. 5L). Indeed, per⁰¹ mutants overexpressing Atg1 responded to iTRF with a significant —14% increase in mean lifespan. That is, while this iTRF-mediated lifespan extension was less than that of genetic controls (˜20%), Atg1 overexpression partially rescued per⁰¹ mutants' lifespan response to iTRF. RU486 alone fed to per⁰¹ mutants on either ad lib or iTRF diets had no effect on lifespan (results not shown).

Thus, in contrast to insulin signaling (FIG. 2D), manipulation of these autophagy regulatory pathways affected iTRF-mediated lifespan.

Thus, taken together, these results supported the hypothesis that clock-dependent enhancement of macroautophagy regulated by Atg1 and Atg8 mediates the effects of iTRF-mediated lifespan extension.

Example 7-Increased Circadian-Regulated Expression of Autophagy-Promoting Genes is Necessary and Sufficient For the Health Benefits of iTRF

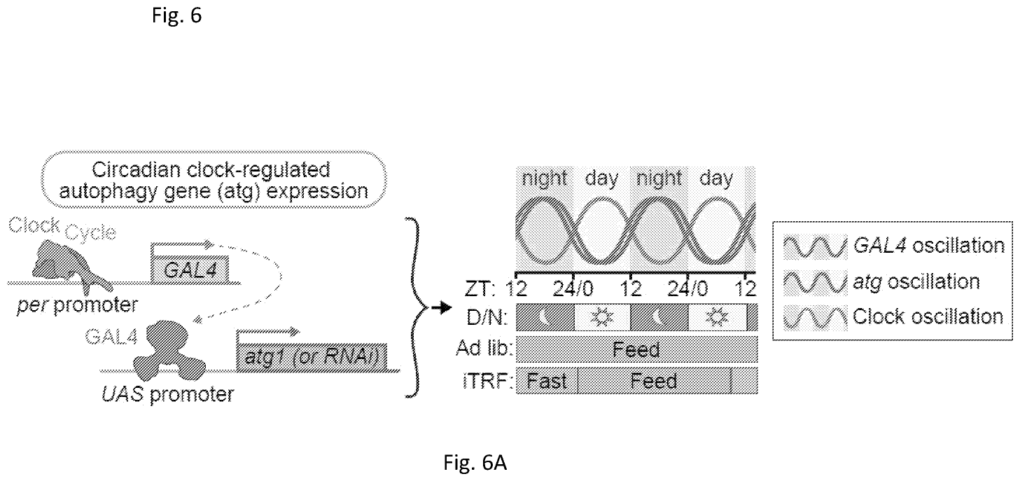

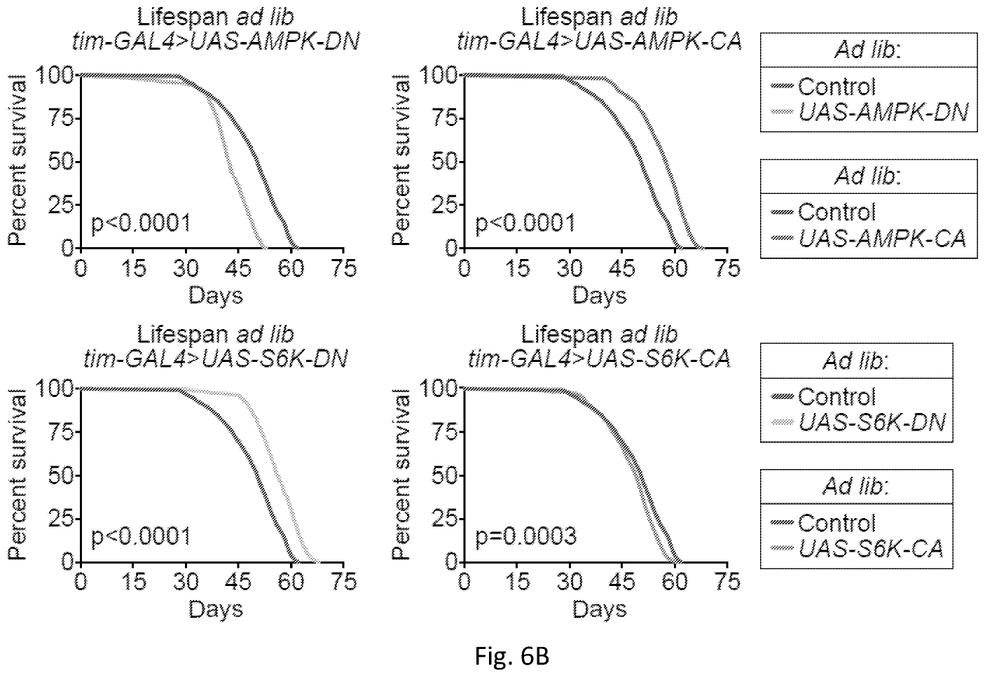

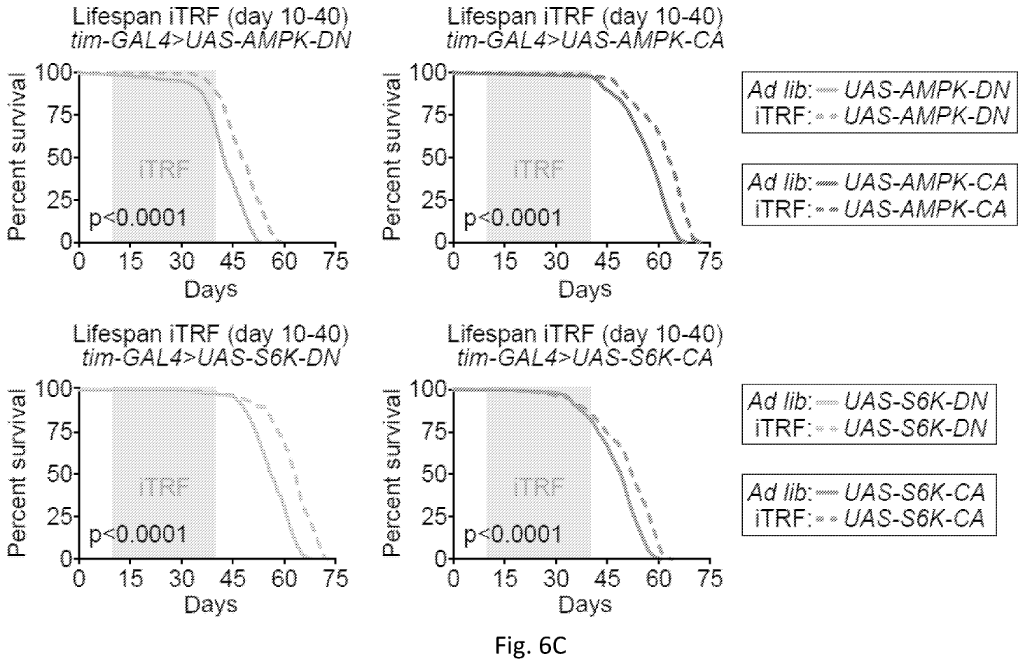

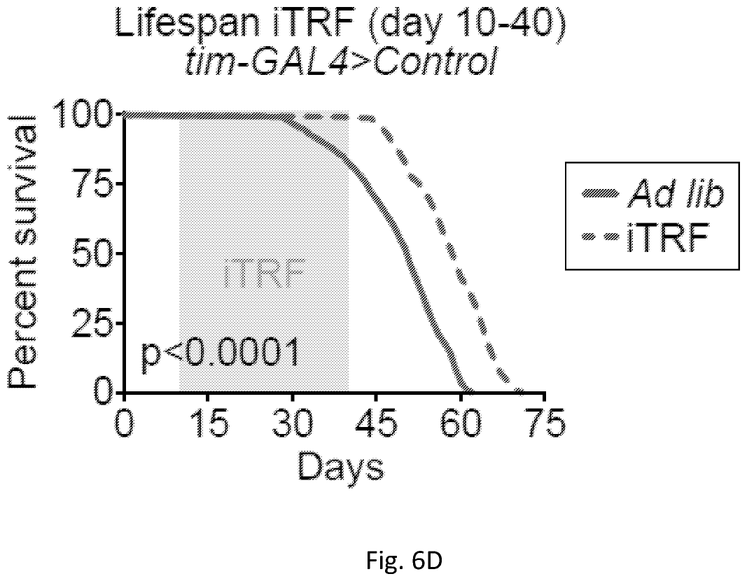

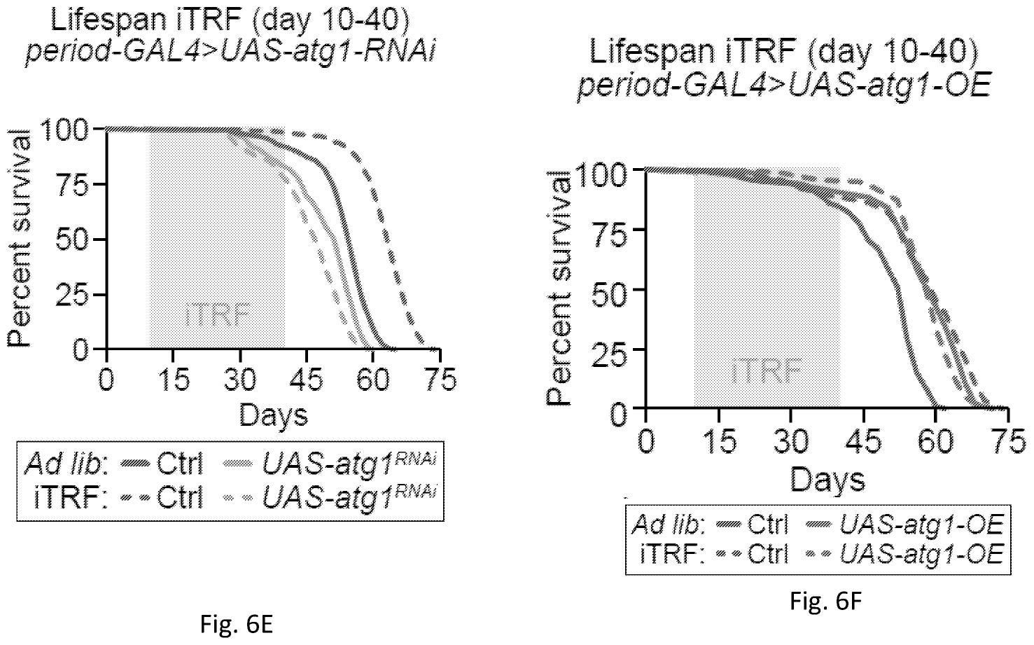

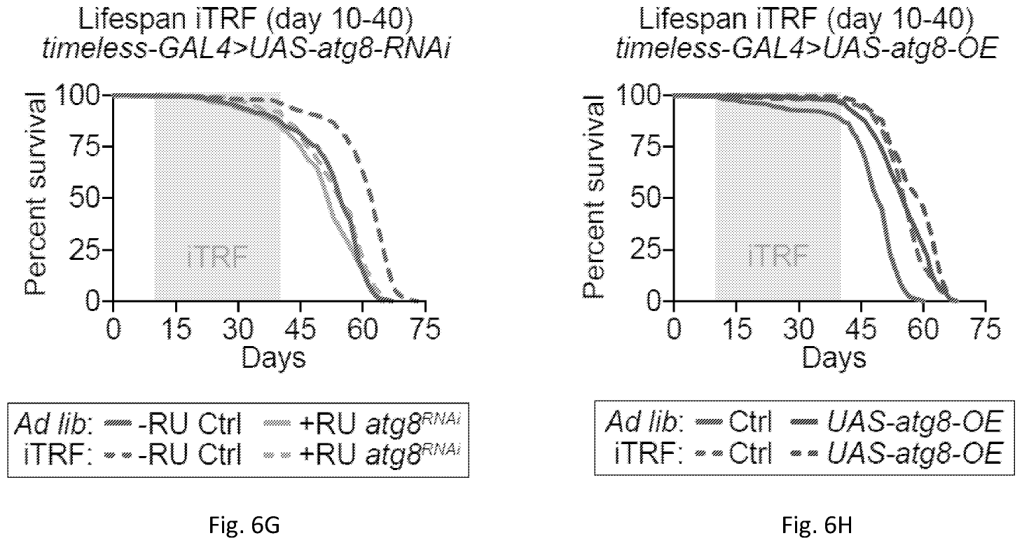

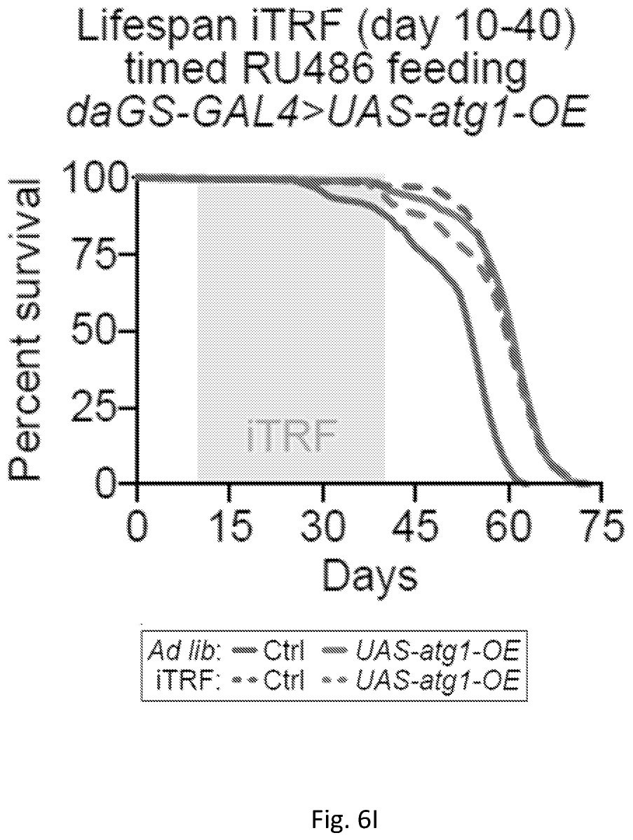

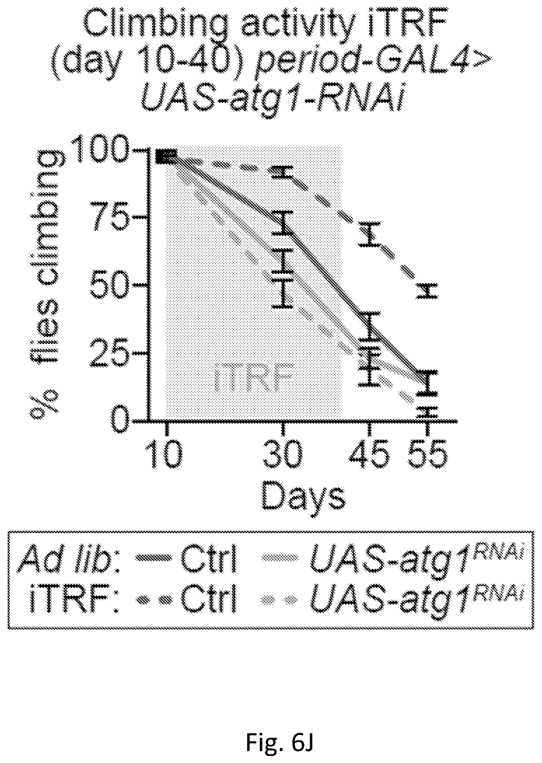

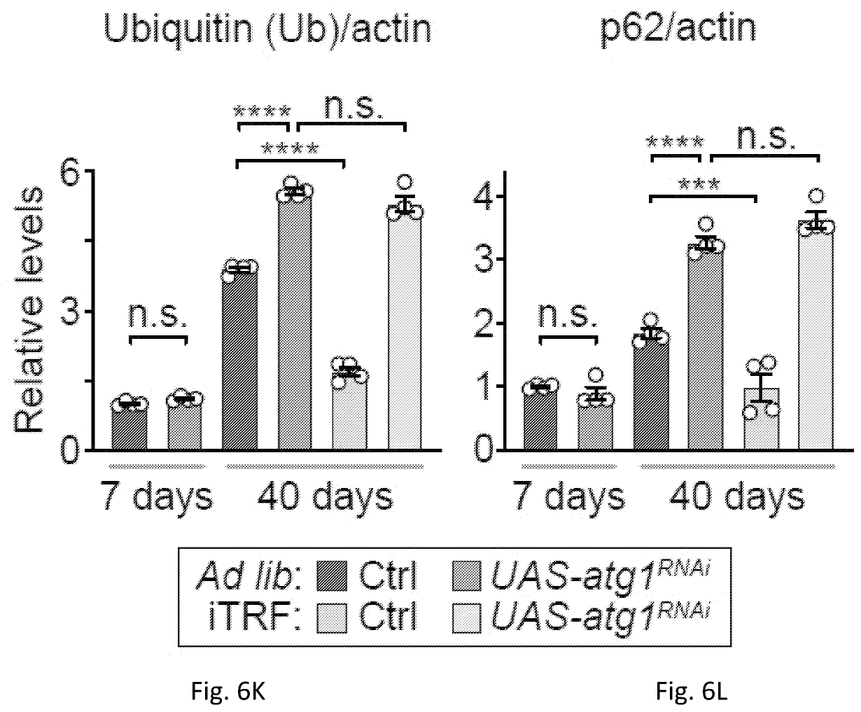

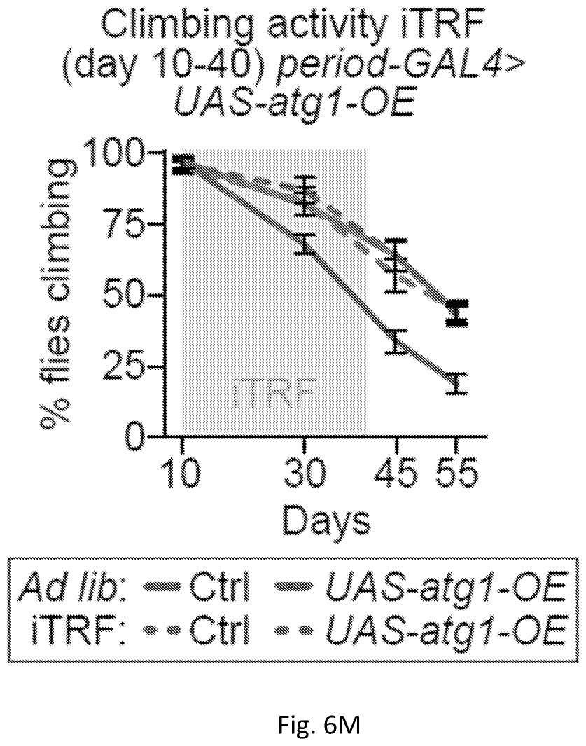

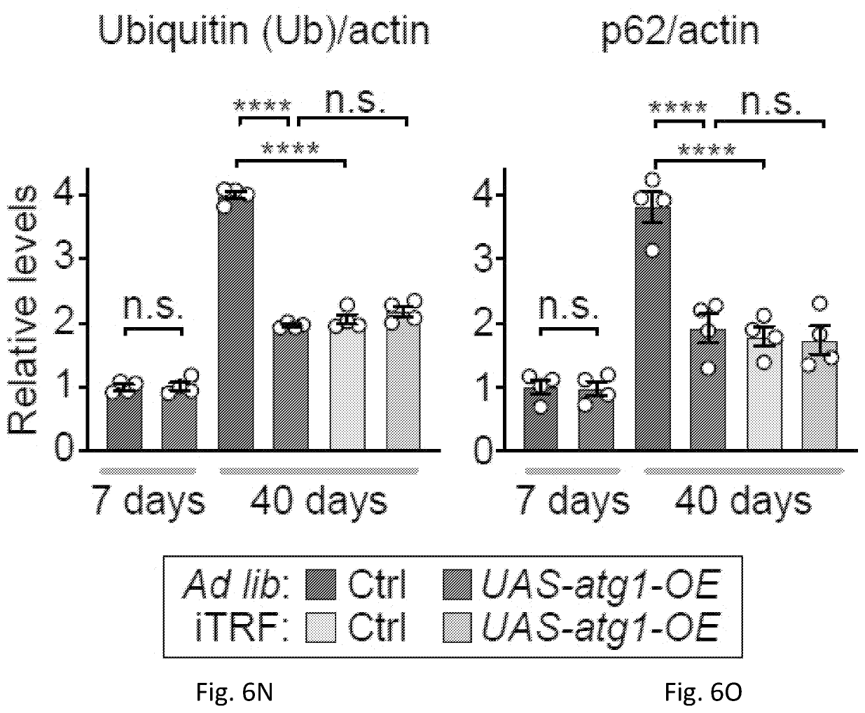

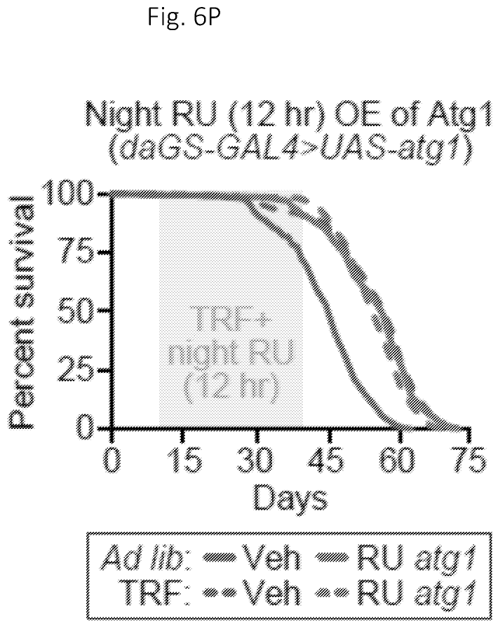

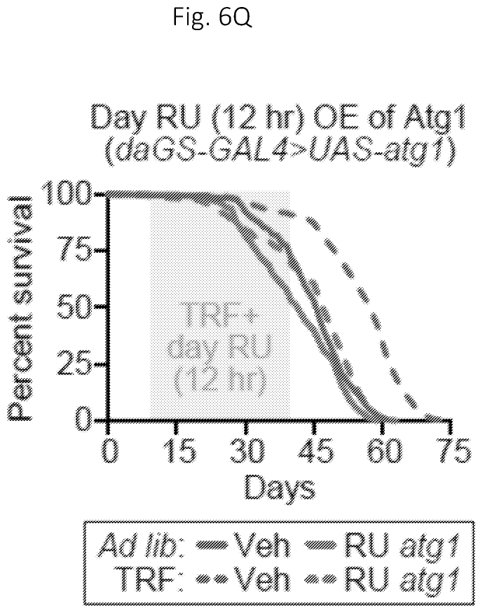

It was next set out to test if circadian manipulation of molecular pathways regulating autophagy impacts iTRF mediated health benefits. To do this, circadian promoters and the UAS GAL4 system were used (FIG. 6A). Based on the expression data, period-GAL4 and timeless-GAL4 were chosen to drive high expression during nighttime fasting periods and low expression during daytime feeding periods. First, to manipulate AMPK and TOR signaling pathways that regulate autophagy, expression of dominant-negative (DN) or constitutively active (CA) constructs of AMPK and S6K were driven (Mirouse et al. 2007; Barcelo et al. 2002). On ad lib control diets, these known aging regulators had expected effects on lifespan, with pro-autophagy manipulations extending lifespan and autophagy inhibition manipulations shortening lifespan (FIG. 6B). On iTRF diets, while DN-S6K did not affect iTRF mediated lifespan extension, DN-AMPK, CA-AMPK, and CA-S6K partially inhibited iTRF-mediated lifespan extension (˜10%) relative to controls (>18%) (FIGS. 6C and 6D). Thus, in contrast to manipulation of insulin signaling (FIG. 2D), manipulation of these autophagy regulatory pathways affected iTRF-mediated lifespan.

Next autophagy gene expression was directly manipulated. Because the timeless-GAL4 driver induced early lethality in combination with the atg1 constructs the weaker driver period-GAL4 22 was used for these experiments. To test if nighttime atg1 expression is required for iTRF-mediated lifespan extension, period-GAL4 was used to drive RNAi-mediated knockdown of atg1. Flies with circadian-regulated knockdown of atg1 showed no iTRF-mediated lifespan extension relative to controls (FIG. 6E). Flies with circadian over-expression of Atg1 not only exhibited iTRF-like lifespan extension without iTRF, while on an ad lib diet, but also exhibited no additional lifespan extension on iTRF (FIG. 6F), suggesting that if circadian-regulated autophagy is already enhanced, iTRF has no effect. These results were consistent with tim-GAL4 overexpression and RNAi knockdown of Atg8 (FIGS. 6G and 6H). Together, these results suggested that enhancement of circadian-regulated autophagy is the major mechanism driving iTRF mediated lifespan extension.

To confirm that these lifespan results are not due to developmental effects, the timed expression of atg1 in adulthood was pharmacologically driven. To mimic the enhanced circadian-regulated expression seen with iTRF, the RU486 inducible daGS-GAL4 was used to drive overexpression of atg1 in flies on ad lib diet during the night/fasting phase (FIG. 6I). Similar to per-GAL4 driving atg1, low level ubiquitous expression of atg1 only during the night/fasting phase was sufficient to extend lifespan similar to that of iTRF.

To confirm that lifespan extension reflects delayed aging, both age related declines in climbing ability and increases in protein aggregation for atg1 RNAi and overexpression mutants and controls on both ad lib and iTRF schedules were tested. Consistent with the requirement of atg1 for lifespan extension, circadian-regulated RNAi knockdown of atg1 prevented iTRF-mediated healthspan extension (FIGS. 6J-6L). Similarly, circadian regulated overexpression of Atg1 caused iTRF-like healthspan extension even on ad lib diet and with no additional improvement on iTRF (FIGS. 6M-6O). Taken together, the data suggested that expression of atg1 in a rhythmic manner is both necessary and sufficient for iTRF-mediated lifespan and healthspan extension.

Because night-specific RU-induced autophagy was sufficient for iTRF-like lifespan extension, day-specific RU-induced autophagy was tested. A night-biased shifted 12:12 TRF regimen with 12-hour fast (ZT9-21) and 12-hour feeding (ZT21-9), without recovery days was developed; the 3-hour offset from lights off removed the evening meal and allowed sufficient fasting for robust lifespan extension similar to iTRF. Similar to this shifted 12:12 TRF, night-specific 12-hr RU induction of atg1 (ZT9-21) produced iTRF-like lifespan extension that resisted further TRF lifespan extension (FIG. 6P). In contrast, day-specific fasting and/or RU treatment (ZT21-9) did not extend lifespan (FIG. 6Q). per01 mutants did not respond to shifted TRF (results not shown). These data suggested that night-specific enhanced atg1 expression is better for iTRF-mediated lifespan extension

Together, our results support the hypothesis that clock-dependent enhancement of macroautophagy mediates the effects of iTRF-mediated lifespan extension. Both circadian regulation and autophagy are key controllers of aging and lifespan (Rubinstein et al. 2011; Duffy et al. 2015). Using genetic manipulations of diverse molecular components known to impact aging and lifespan, circadian clock components (Timeless, Period, Cycle, and Clock) and essential autophagy components Atg 1 and Atg8 were identified as both necessary and sufficient for the anti-aging, lifespan-extending benefits of iTRF.

### REFERENCES

Allada and Chung, Circadian organization of behavior and physiology in Drosophila. Annu Rev Physiol 72, 605-624 (2010).
Barcelo and Stewart, Altering Drosophila S6 kinase activity is consistent with a role for S6 kinase in growth. Genes 34, 83-85 (2002).
Catterson et al., Short-Term, Intermittent Fasting Induces Long-Lasting Gut Health and TOR-Independent Lifespan Extension. Curr Biol 28, 1714-1724 (2018).
Chaix et al., Time-restricted feeding is a preventative and therapeutic intervention against diverse nutritional challenges. Cell Metab 20, 991-1005 (2014).
Claesson et al., Comparison of two next-generation sequencing technologies for resolving highly complex microbiota composition using tandem variable 16S rRNA gene regions. Nucleic Acids Res 38, e200-e200 (2010).
Copeland et al., Extension of Drosophila Life Span by RNAi of the Mitochondrial Respiratory Chain. Current Biology 19, 1591-1598 (2009).
Delventhal et al., Dissection of central clock function in Drosophila through cell specific CRISPR-mediated clock gene disruption. eLife 8, e48308, (2019).
Duffy et al., Aging and Circadian Rhythms. Sleep Med Clin 10, 423-434 (2015). Gill et al., Time-restricted feeding attenuates age-related cardiac decline in Drosophila. Science 347, 1265-1269 (2015).
Ja et al., Prandiology of Drosophila and the CAFE assay. Proc Natl Acad Sci USA 104, 8253-8256 (2007).
Kalfalah et al., Crosstalk of clock gene expression and autophagy in aging. Aging 8, 1876-1895 (2016).
Li et al., Intermittent Fasting Promotes White Adipose Browning and Decreases Obesity by Shaping the Gut Microbiota. Cell Metab 26, 672-685 (2017).
Longo and Panda, Fasting, Circadian Rhythms, and Time-Restricted Feeding in Healthy Lifespan. Cell Metab 23, 1048-1059 (2016).
Ma et al., Circadian autophagy rhythm: a link between clock and metabolism? Trends Endocrinol Metab 23, 319-325 (2012).
Manoogian and Panda, Circadian rhythms, time-restricted feeding, and healthy aging. Ageing Research Reviews 39, 59-67 (2017).

Mirouse et al., LKB1 and AMPK maintain epithelial cell polarity under energetic stress. Journal of Cell Biology 177, 387-392 (2007).

Murphy et al., Simultaneous measurement of sleep and feeding in individual Drosophila. Nat Protoc 12, 2355-2366 (2017).
Murphy et al. Postprandial sleep mechanics in Drosophila. eLife 5, e19334 (2016).
Panda, Circadian physiology of metabolism. Science 354, 1008-1015, (2016).
Partridge et al., Ageing in Drosophila: the role of the insulin/Igf and TOR signalling network. Exp Gerontol 46, 376-381 (2011).
Rana et al., Parkin overexpression during aging reduces proteotoxicity, alters mitochondrial dynamics, and extends lifespan. Proc Natl Acad Sci USA 110, 8638 (2013).
Rera et al., Intestinal barrier dysfunction links metabolic and inflammatory markers of aging to death in Drosophila. Proc Natl Acad Sci USA 109, 21528 (2012).
Rera et al., Modulation of longevity and tissue homeostasis by the Drosophila PGC-1 homolog. Cell Metab 14, 623-634 (2011).
Rubinsztein et al., Autophagy and Aging. Cell 146, 682-695 (2011).
Schneider et al., NIH Image to ImageJ: 25 years of image analysis. Nat Methods 9, 671-675 (2012).
Tricoire et al., The steroid hormone receptor EcR finely modulates Drosophila lifespan during adulthood in a sex-specific manner. Mechanisms of Ageing and Development 130, 547-552 (2009).
Ulgherait et al., Circadian regulation of mitochondrial uncoupling and lifespan. Nature Communications 11, 1927 (2020).
Ulgherait et al., Dietary Restriction Extends the Lifespan of Circadian Mutants tim and per. Cell Metab 24, 763-764 (2016).
Villanueva et al., Time-restricted feeding restores muscle function in Drosophila models of obesity and circadian-rhythm disruption. Nature Communications 10, 2700 (2019).

1-17. (canceled)

18. A method of increasing and/or extending lifespan and/or healthspan and/or delaying aging in a subject by administering a therapeutically effective amount of an agent which activates or enhances circadian-regulated autophagy.

19. The method of claim 18, wherein the agent is administered during the resting phase of the subject's circadian cycle.

20. The method of claim 18, wherein the agent is selected from the group consisting of chemicals, pharmaceuticals, biologics, small organic molecules, antibodies, nucleic acids, peptides, and proteins.

21. The method of claim 18, wherein the agent which increases, activates or enhances a protein selected from the group consisting of human UNC-51-like kinase (ULK1), adenosine monophosphate protein kinase (AMPK) and microtubule-associated protein, light chain 3 (LC3) or a therapeutically effective amount of an agent which decreases or inhibits ribosomal protein S6 kinase beta-1 (S6K) protein.

22. The method of claim 21, wherein the agent is selected from the group consisting of 5-Aminoimidazole-4-carboxamide 1-b-D-ribofuranoside (AICAR), Metformin HCl, LYN-1604, BL-918, (rac)-BL-918, and rapamycin.

23. The method of claim 21, wherein the agent is a transgene or nucleic acid encoding a protein selected from the group consisting of ULK1, AMPK, and LC3, or a variant, mutant, fragment, homologue, or derivative thereof, or a vector or composition comprising a nucleic acid or transgene which encodes a protein selected from the group consisting of human ULK1, AMPK, and LC3, or a variant, mutant, fragment, homologue, or derivative thereof.

24. The method of claim 21, wherein the agent is a vector or composition comprising a nucleic acid which inhibits or targets S6K.

25. The method of claim 18, wherein the subject is over 50 years of age.

26. A method of preventing and/or treating an age-related illness in a subject by administering a therapeutically effective amount of an agent which activates or enhances circadian-regulated autophagy.

27. The method of claim 26, wherein the age-related illness is a condition or disease of the muscles.

28. The method of claim 26, wherein the age-related illness is a condition or disease of the intestinal tract.

29. The method of claim 26, wherein the agent is administered during the resting phase of the subject's circadian cycle.

30. The method of claim 26, wherein the agent is selected from the group consisting of chemicals, pharmaceuticals, biologics, small organic molecules, antibodies, nucleic acids, peptides, and proteins.

31. The method of claim 26, wherein the agent which increases, activates or enhances a protein selected from the group consisting of human UNC-51-like kinase (ULK1), adenosine monophosphate protein kinase (AMPK) and microtubule-associated protein, light chain 3 (LC3) or a therapeutically effective amount of an agent which decreases or inhibits ribosomal protein S6 kinase beta-1 (S6K) protein.

32. The method of claim 31, wherein the agent is selected from the group consisting of 5-Aminoimidazole-4-carboxamide 1-b-D-ribofuranoside (AICAR), Metformin HCl, LYN-1604, BL-918, (rac)-BL-918, and rapamycin.

33. The method of claim 31, wherein the agent is a transgene or nucleic acid encoding a protein selected from the group consisting of ULK1, AMPK, and LC3, or a variant, mutant, fragment, homologue, or derivative thereof, or a vector or composition comprising a nucleic acid or transgene which encodes a protein selected from the group consisting of human ULK1, AMPK, and LC3, or a variant, mutant, fragment, homologue, or derivative thereof.

34. The method of claim 31, wherein the agent is a vector or composition comprising a nucleic acid which inhibits or targets S6K.

35. The method of claim 26, wherein the subject is over 50 years of age.
